(19) United States
(12) Patent Application Publication
(10) Pub. No.: US 2026/0053750 A1
(43) Pub. Date: Feb. 26, 2026

(54) INHALABLE COMPOSITIONS OF CDK9 INHIBITORS

(71) Applicants: Matthew OWEN, Oakland, CA (US); Tom YARBROUGH, Davis, CA (US); THE REGENTS OF THE UNIVERSITY OF CALIFORNIA, Oakland, CA (US); TESIO PHARMACEUTICALS, INC., Davis, CA (US)

(72) Inventors: Matthew OWEN, Oakland, CA (US); Tom YARBROUGH, Davis, CA (US); Dominik HAUDENSCHILD, Oakland, CA (US); Jasper YIK, OAKLAND, CA (US); Gang-Yu LIU, OAKLAND, CA (US); Ching-Hsien CHEN, OAKLAND, CA (US); Reen WU, OAKLAND, CA (US); Lark COFFEY, OAKLAND, CA (US); Mei-chang KUO, DAVIS, CA (US)

(21) Appl. No.: 19/104,353

(22) PCT Filed: Aug. 17, 2023
(86) PCT No.: PCT/US2023/072365

§ 371 Date: Feb. 17, 2025

Related U.S. Application Data
(60) Provisional application No. 63/371,721, filed on Aug. 17, 2022.

(51) Int. Cl. A61K 9/50; A61K 31/453; A61K 45/06

(52) U.S. Cl. CPC A61K 9/5015; A61K 9/501; A61K 9/5089; A61K 31/453; A61K 45/06

(57) ABSTRACT

The present disclosure describes a formulation for the delivery of CDK9 inhibitors.

### CROSS-REFERENCES TO RELATED APPLICATIONS

This application claims priority to U.S. Provisional Application No. 63/371,721, filed Aug. 17, 2022, which is incorporated herein in its entirety for all purposes.

### STATEMENT AS TO RIGHTS TO INVENTIONS MADE UNDER FEDERALLY SPONSORED RESEARCH OR DEVELOPMENT

This invention was made with Government support from the Department of Defense via the Congressionally Directed Medical Research Programs under Grant Number PR200947. The Government has certain rights in the invention.

### BACKGROUND OF THE INVENTION

Inflammation in the lungs is a principle cause of diseases and even death including lung fibrosis, lung cancer, and more recently lung failure due to COVID-19. Therefore, anti-inflammatory represents an important target of medical interventions. Prior work done by us and others have identified a potent anti-inflammation therapeutic, flavopiridol, a well-known small molecule inhibitor for cyclin dependent kinase 9 inhibitor (CDK9). It was originally designated by the Food and Drug Administration (FDA) as an orphan drug for treatment of rare leukemias. Our team revealed that flavopiridol inhibits activation of primary response genes, thus oppress down-stream inflammation of cells and tissues. Therefore. Flavopiridol has been tested in the treatment of illnesses ranging from cancer, viral and post-traumatic-osteoarthritis (PTOA). Given its efficacy, Flavopiridol has been identified as a potentially promising candidate to treat illnesses such as lung cancer or coronavirus disease 2019 (COVID-19). In most cases, anti-inflammation medicines were typically dosed systemically such as intravenous (i.v.) or oral delivery. High dosage and severe side effects often occurred due to systemic delivery. Thus, local delivery would be more desirable such as pulmonary delivery by inhalation. The local delivery of medicines to the lung is rapid in therapeutic efficacy and has been shown to improve the percentage of the bioavailable drug for treatment. Additional advantages of inhalation delivery such as a simple dry powder inhaler (DPI) include non-invasive, high safety and fast in reaching targets.

Inhalable dry particles are typically produced using either jet milling or spray drying techniques. Spray drying involves the rapid drying of liquid formulations into dry powders that are filled into either gelatin capsules or blister packs for use in dry powder inhalers. While it produces high throughput, even mini spray-drying apparatus such as the Buchi Mini Dryer B290 require several hundred milligrams of often costly drug material to produce a single sample and those mini dryers are also expensive (˜$35K). Thus, using spray-drying in research laboratories to carry out initial research and development (R&D) efforts such as optimize formulation and particle size, and understand mechanism, would be challenging and costly. This work introduces a simple lab-constructed table-top device that enables the production of flavopiridol-loaded inhalable ultra-small particles for pulmonary delivery. The particles produced meet the requirements of physio-chemical property, inhalability, and release profiles needed for pulmonary delivery. The anti-inflammation activity has also been demonstrated via in vitro testing. The device and conditions introduced in this work could be utilized by researchers in their R&D efforts in drug delivery. The particles produced will be tested in vivo for treatments of lung inflammation, and as new therapeutic means to treat lung cancer, pulmonary fibrosis, and COVID-19.

### BRIEF SUMMARY OF THE INVENTION

In one embodiment, the present invention provides a microparticle composition comprising a plurality of microparticles, wherein each microparticle comprises: a hydrophobic amino acid or a hydrophobic peptide, or combinations thereof: a lipid; and a cyclin-dependent kinase 9 (CDK9) inhibitor.

In another embodiment, the present invention provides a composition for use in treating inflammation or respiratory fibrosis in a subject in need thereof, wherein the composition comprises a cyclin-dependent kinase 9 (CDK9) inhibitor.

In another embodiment, the present invention provides a method of preparing a plurality of particles of the present invention, comprising
sonicating a first reaction mixture comprising L-isoleucine and 1,2-dipalmitoyl-sn-glycero-3-phosphocholine (DPPC), water, and ethanol, wherein the isoleucine and DPPC are present in a ratio of about 9:1 (w/w), and wherein the ethanol:water ratio is about 70:30 (v/v), to prepare a 0.3% (w/v) feed mixture;
applying the feed mixture to a microfluidic piezo array to form a spray of micronized droplets; and
drying the droplets to afford the plurality of particles.

In another embodiment, the present invention provides a liquid composition comprising: a citric acid buffer having a pH of from 2 to 7; and a cyclin-dependent kinase 9 (CDK9) inhibitor.

In another embodiment, the present invention provides a method of administering a therapeutically effective amount of a CDK9 inhibitor to a subject in need thereof, comprising administering to the subject a microparticle composition of the present invention, or a liquid composition of the present invention, via respiratory administration.

In another embodiment, the present invention provides a method of treating inflammation in a subject in need thereof, comprising administering a therapeutically effective amount of a microparticle composition of the present invention, or a liquid composition of the present invention, to the subject via respiratory administration, thereby treating the inflammation.

In another embodiment, the present invention provides a method of treating respiratory fibrosis in a subject in need thereof, comprising administering a therapeutically effective amount of a microparticle composition of the present invention, or a liquid composition of the present invention, to the subject via respiratory administration, thereby treating the respiratory fibrosis.

In another embodiment, the present invention provides a method of treating inflammation or respiratory fibrosis in a subject in need thereof, comprising administering a therapeutically effective amount of a composition comprising a cyclin-dependent kinase 9 (CDK9) inhibitor.

### BRIEF DESCRIPTION OF THE DRAWINGS

FIG. 1 shows a schematic diagram of the Microfluidic Piezo and Cyclone Apparatus (MPCA) for the formation of inhalable particles.

FIG. 2 shows FTIR spectra of excipient materials of (A) L-isoleucine, (B) DPPC, displayed in conjunction with the ILDF (L-IsoLeucine, DPPC, Flavopiridol) microparticles (C).

FIG. 3A to 3D shows SEM imaging from four experiments during our investigations to determine our formulation. FIG. 3A shows excipients include L-isoleucine:DPPC=90:10 (w:w) in a mixed solvent of 70% (v/v) ethanol/water. FIG. 3B shows L-isoleucine:DPPC:CaCl₂)=89:10:0.75 in a mixed solvent with the same mixing as that in FIG. 3A. FIG. 3C shows L-isoleucine:DPPC:Glucose=10:10:80 in a mixed solvent with the same mixing as that in FIG. 3A. FIG. 3D shows L-isoleucine:DPPC:Glucose=10:10:80 in a mixed solvent of 80% (v/v) ethanol:water. Scale bars=20 μm.

FIG. 4 shows the release profile of flavopiridol from the microparticles.

FIG. 5A to 5C show stability data for the particles, including at 4° C. (FIG. 5A), 24° C. (FIG. 5B), and at 37° C. (FIG. 5C).

FIG. 6 shows luminesence reading for samples treated with TNF-α or flavopiridol.

FIG. 7A to FIG. 7C show testing of the dispersity of the particles using a device (FIG. 7A), with the particles demonstrating high dispersity (FIG. 7B), while the corresponding glucose particles showed little to no dispersability (FIG. 7C).

FIG. 8A to 8E show results of administration of flavopiridol in a lung fibrosis model. The administration of flavopiridol showed a statistically significant difference in survival rate as compared to the BLM+ vehicle control (FIG. 8A). The flavopiridol treated mice generally maintained a higher body weight than the BLM+ vehicle control (FIG. 8B) and a statistically significant lower level of hydroxyproline (FIG. 8C). Lung tissue sections from the treated animals were stained with Masson's trichome staining protocol and exemplary images are shown in FIG. 8D. Quantitation of the staining is shown in FIG. 8E.

FIG. 9 shows the body weights of mice measured at the days indicated (mean plus/minus SE).

FIG. 10 shows the Kaplan-Meier analysis of the overall survival rates of vehicle- and inhaled flavopiridol-treated mice with a single dose of bleomycin (n=12).

FIG. 11 shows hydroxyproline content in the right lung homogenate samples detected by the hydroxyproline ELISA assay. Data are presented as mean with individual values (mean plus/minus SE. *p<0.05 vs Saline+Vehicle).

FIG. 12 shows representative hematoxylin and eosin (H&E) and Masson's trichrome staining of lung tissue from vehicle-treated and inhaled flavopiridol-treated mice with a single dose of bleomycin challenge (Left). Semiquantitative fibrosis evaluation of fibrotic lesions on H&E-stained sections of mouse lung (top) and positive staining on histological Masson's trichrome-stained sections of mouse lung (Right). Fibrosis score is presented as the percentage of the positive staining area per high-powered field. Quantitative analysis of 6-12 high-powered fields per lung was performed by using ImageJ software (mean±SE, *p<0.05).

### DETAILED DESCRIPTION OF THE INVENTION

### I. General

The present invention describes microparticles of L-isoleucine, 1,2-dipalmitoyl-sn-glycero-3-phosphocholine (DPPC), and a cyclin-dependent kinase 9 (CDK9) inhibitor such as flavopiridol.

### II. Definitions

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Naturally occurring amino acids include Alanine (A), Glycine (G). Aspartic acid (D), Glutamic acid (E), Asparagine (N), Glutamine (Q), Arginine (R). Lysine (K), Isoleucine (I). Leucine (L). Methionine (M), Valine (V), Phenylalanine (F), Tyrosine (Y), Tryptophan (W), Serine(S), Threonine (T), and Cysteine (C).

“Amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified backbones, but retain the same basic chemical structure as a naturally occurring amino acid.

“Unnatural amino acids” are not encoded by the genetic code and can, but do not necessarily have the same basic structure as a naturally occurring amino acid. Unnatural amino acids include, but are not limited to azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine, 2,4-diaminoisobutyric acid, desmosine, 2,2′-diaminopimelic acid, 2,3-diaminopropionic acid, N-ethylglycine, N-ethylasparagine, homoproline, hydroxylysine, allo-hydroxy lysine, 3-hydroxyproline, 4-hydroxyproline, isodesmosine, allo-isoleucine, N-methylalanine, N-methylglycine, N-methylisoleucine, N-methylpentylglycine, N-methylvaline, naphthalanine, norvaline, ornithine, pentylglycine, pipecolic acid and thioproline.

“L-isoleucine” refers to the compound having CAS No. 73-32-5, (2S,3S)-2-Amino-3-methylpentanoic acid.

“Peptide” refers to a series of amino acid residues covalently linked together. Peptides can includes 2, 3, 4 or more amino acid residues of any amino acid. A hydrophobic peptide is a peptide including a majority of hydrophobic amino aicds. Representative hydrophobic peptides include trileucine.

“Trileucine” or “Leu-Leu-Leu” or “L-L-L” refers to the compound having CAS No. 10329-75-6.

“Lipid” refers to small molecules having hydrophobic or amphiphilic properties and are useful for preparation of vesicles, micelles and liposomes. Lipids include, but are not limited to, fats, waxes, fatty acids, cholesterol, phospholipids, monoglycerides, diglycerides and triglycerides. The lipid moiety can include several fatty acid groups using branching groups such as lysine and other branched amines. Phospholipids or phosphine lipids refers to lipids having the moiety P(O)₂(OR)₂, wherein one R group is hydrophilic and one R group is hydrophilic.

“Pharmaceutically acceptable salt” refers to acid or base salts of the compounds used in the methods of the present invention. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17^{th} ed., Mack Publishing Company, Easton, Pa., 1985, which is incorporated herein by reference.

Pharmaceutically acceptable salts of the acidic compounds of the present invention are salts formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

Similarly acid addition salts, such as of mineral acids, organic carboxylic and organic sulfonic acids, e.g., hydrochloric acid, methanesulfonic acid, maleic acid, are also possible provided a basic group, such as pyridyl, constitutes part of the structure.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

“CDK” refers to cyclin-dependent kinase. CDK9 is cyclin-dependent kinase 9.

“Derivative” of a CDK9 inhibitor is an ester, amide, or prodrug of the CDK9 inhibitor, where the ester, amide, or prodrug substituent is cleaved or hydrolyzed after administration to a subject.

“Microparticle” refers to a particle of the present invention having a diameter between about 0.5 μm and about 10 μm.

“Pharmaceutically acceptable carrier” refers to a typically inert substance used as a diluent or vehicle for a drug such as a therapeutic agent. The term also encompasses a typically inert substance that imparts cohesive qualities to the composition. Typically, the physiologically acceptable carriers are present in liquid form. Examples of liquid carriers include physiological saline, phosphate buffer, normal buffered saline, water, buffered water, saline, glycine, glycoproteins to provide enhanced stability (e.g., albumin, lipoprotein, globulin, etc.), and the like. Since physiologically acceptable carriers are determined in part by the particular composition being administered as well as by the particular method used to administer the composition, there are a wide variety of suitable formulations of pharmaceutical compositions of the present invention (See, e.g., Remington's Pharmaceutical Sciences, 17.sup.th ed., 1989).

“Sonicate” or “sonicating” refers to application of sound energy to a solution or mixture.

“Reaction mixture” refers to a mixture of at least two distinct species.

“Drying” refers to removal of moisture or solvent from a mixture.

“Power” refers to electrical power.

“Buffer” or “buffering agent” refers to any inorganic or organic acid or base that resists changes in pH and maintains the pH around a desired point. Buffering agents useful in the present invention include, but are not limited to, sodium hydroxide, dibasic sodium phosphate anhydrous, and mixtures thereof. One of skill in the art will appreciate that other buffering agents are useful in the present invention.

“Treat”, “treating” and “treatment” refers to any indicia of success in the treatment or amelioration of an injury, pathology, condition, or symptom (e.g., pain), including any objective or subjective parameter such as abatement; remission; diminishing of symptoms or making the symptom, injury, pathology or condition more tolerable to the patient; decreasing the frequency or duration of the symptom or condition; or, in some situations, preventing the onset of the symptom. The treatment or amelioration of symptoms can be based on any objective or subjective parameter; including, e.g., the result of a physical examination.

“Administering” refers to oral administration, administration as respiratory, inhalation, nasal, a suppository, topical contact, parenteral, intravenous, intraperitoneal, intramuscular, intralesional, intranasal or subcutaneous administration, or intrathecal administration, to the subject.

“Subject” as used herein refers to a mammal, which can be a human or a non-human mammal, for example a companion animal, such as a dog, cat, rat, or the like, or a farm animal, such as a horse, donkey, mule, goat, sheep, pig, or cow, and the like.

“Therapeutically effective amount” refers to the amount of the microparticles of the invention sufficient to suppress undesirable inflammation and to eliminate or at least partially arrest symptoms and/or complications. Specifically, a therapeutically effective amount is the amount sufficient to suppress expression of primary response genes such as IL-1β and IL-6 to no more than 50%, 40%, 30%, 20%, 10%, 5%, or 1% of the otherwise expected gene activity. Amounts effective for this use will depend on. e.g., the inhibitor composition, the manner of administration, the stage and severity of the disease being treated, the weight and general state of health of the patient, and the judgment of the prescribing physician. In practice, the amount of CDK9 inhibitor required for a therapeutic effect in the method of the invention will be less than the amount required for systemic administration, due to the local nature of the microparticle drug release. The microparticles of the invention can be administered chronically or acutely to reduce, inhibit or prevent inflammation, cartilage degradation, and post traumatic osteoarthritis.

“Lung fibrosis”, “pulmonary fibrosis”, or “respiratory fibrosis” refers to a condition involving scarring of the lungs leading to shortness of breath, cough, tiredness, weight loss, and clubbing of the fingernails.

“Sustained release” refers to the release of CDK9 inhibitor over an extended period of time after administration, generally between about 1 hour and about 30-60 days.

### III. CDK9 Composition

The present invention provides compositions for treating inflammation. In some embodiments, the present invention provides a composition comprising a cyclin-dependent kinase 9 (CDK9) inhibitor.

a. Cyclin-Dependent Kinase 9 (CDK9) Inhibitor

Provided herein are formulations of therapeutic agents that target Cdk9 kinase activity using existing small-molecule inhibitors of CDK9. The formulations provided herein are suitable with flavopiridol, voruciclib and the class of CDK9 inhibitors structurally related to flavopiridol and voruciclib such that the inhibitor is delivered with appropriate overall release potential and release kinetics to the affected site of a subject, such as an injured tissue or cell type.

In some embodiments, the CDK9 inhibitor is flavopiridol, or an ester, prodrug, or pharmaceutically acceptable salt thereof. In some embodiments, the CDK9 inhibitor is flavopiridol, SNS-032, or voruciclib, or a derivative thereof, or a pharmaceutically acceptable salt thereof. In some embodiments, the CDK9 inhibitor is flavopiridol, SNS-032, or a pharmaceutically acceptable salt thereof. In some embodiments, the CDK9 inhibitor is flavopiridol, SNS-032, or voruciclib. In some embodiments, the CDK9 inhibitor is flavopiridol, or a pharmaceutically acceptable salt thereof. In some embodiments, the CDK9 inhibitor is flavopiridol. In some embodiments, the CDK9 inhibitor is flavopiridol HCl.

In some embodiments, the CDK9 inhibitor is flavopiridol, or an ester, prodrug, or pharmaceutically acceptable salt thereof. In some embodiments, the CDK9 inhibitor is flavopiridol, or a derivative or salt thereof. In some embodiments, the CDK9 inhibitor is flavopiridol (IUPAC name: 2-(2-chlorophenyl)-5,7-dihydroxy-8-[(3S,4R)-3-hydroxy-1-methyl-4-piperidinyl]-4-chromenone: CAS #146426-40-6), having a structure of:

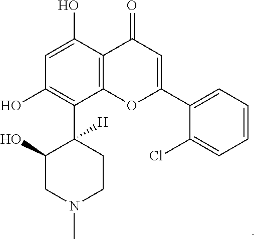 [SMILES: CN1CC[C@H](c2c(O)cc(O)c3c(=O)cc(-c4ccccc4Cl)oc23)[C@H](O)C1]

CDK9 inhibitors such as flavopiridol broadly and efficiently suppress the transcriptional activation of primary response genes, which includes inflammatory genes (such as IL-1. TNF. IL-6, iNOS, etc.) and matrix degrading enzymes (MMPs, ADAMTS, etc.). However, flavopiridol is rapidly metabolized and degraded, and has a short in-vivo half-life of under 6 hours. As a small molecule (˜400 Da), it rapidly diffuses from the site of administration, and is therefore generally given as a systemic administration. Provided herein are formulations of a CDK9 inhibitor and a PLGA polymer, wherein the CDK9 inhibitor is encapsulated in a particle of appropriate size and with appropriate release potential and release kinetics such that the CDK9 inhibitor is provided in a therapeutically effective amount over a duration to treat an injury, reduce inflammation, ameliorate symptoms and/or prevent further damage to an injured tissue of a subject.

In some embodiments, the CDK9 inhibitor is SNS-032, or a prodrug, or a pharmaceutically acceptable salt thereof. In some embodiments, the CDK9 inhibitor is SNS-032, or a salt thereof. In some embodiments, the CDK9 inhibitor is SNS-032, having the structure:

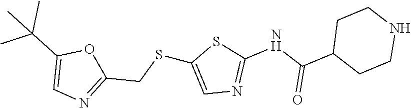 [SMILES: CC(C)(C)c1cnc(CSc2cnc(NC(=O)C3CCNCC3)s2)o1]

In some embodiments, the CDK9 inhibitor is voruciclib, or an ester, prodrug, or pharmaceutically acceptable salt thereof. In some embodiments, the CDK9 inhibitor is voruciclib, or a derivative or salt thereof. In some embodiments, the CDK9 inhibitor is voruciclib, having the structure:

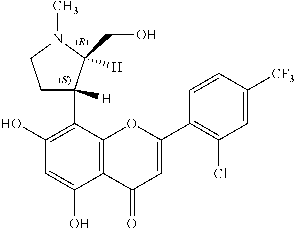

Another CDK9 inhibitor is dinaciclib. Dinaciclib is not encapsulated effectively or released appropriately in microparticles of the invention:

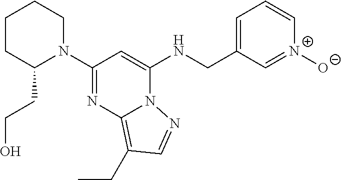 [SMILES: CCc1cnn2c(NCc3ccc[n+]([O-])c3)cc(N3CCCC[C@H]3CCO)nc12]

Provided herein are formulations of a CDK9 inhibitor wherein the CDK9 inhibitor is SNS-32, voruciclib, or flavopiridol, and a PLGA polymer such that the CDK9 inhibitor, is encapsulated in a microparticle of appropriate size and with appropriate release potential and release kinetics such that the CDK9 inhibitor is provided in a therapeutically effective amount over a duration to treat an injury, reduce inflammation, ameliorate symptoms and/or prevent further damage to an injured tissue of a subject.

Provided are also pharmaceutically acceptable salts, hydrates, solvates, tautomeric forms, polymorphs, and prodrugs of the CDK9 inhibitors described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The compounds described herein may be prepared and/or formulated as pharmaceutically acceptable salts, or when appropriate as a free base. “Pharmaceutically acceptable salts” are non-toxic salts of a free base form of a compound that retain the desired pharmacological activity of the free base. These salts may be derived from inorganic or organic acids or bases. For example, a compound that contains a basic nitrogen may be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxy benzoates, methoxy benzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxy butyrates, glycolates, tartrates, and mandelates. Other suitable pharmaceutically acceptable salts are found in Remington: The Science and Practice of Pharmacy, 21^{st} Edition, Lippincott Williams and Wilkins, Philadelphia, PA, 2006.

Examples of pharmaceutically acceptable salts of the compounds disclosed herein also include salts derived from an appropriate base, such as an alkali metal (for example, sodium, potassium), an alkaline earth metal (for example, magnesium), ammonium and NX₄⁺ (wherein X is C₁-C₄ alkyl). Also included are base addition salts, such as sodium or potassium salts.

The CDK9 inhibitor can be present in the composition in any suitable amount. For example, the CDK9 inhibitor can be present in an amount of from 0.01 to 10% (w/w), or from 0.01 to 5%, or from 0.01 to 1%, or from 0.1 to 1%, or from 0.1 to 0.5%, or from 0.1 to 0.3% (w/w). The CDK9 inhibitor can be present in the microparticles in an amount of about 0.01% (w/w), or 0.05, 0.1, 0.11, 0.12, 0.13, 0.14, 0.15, 0.16, 0.17, 0.18, 0.19, 0.2, 0.21, 0.22, 0.23, 0.24, 0.25, 0.3, 0.35, 0.4, 0.45, or about 0.5% (w/w).

The CDK9 inhibitor can be present in a ratio relative to the lipid. For example, the lipid can be present in a ratio to the CDK9 inhibitor of from 1000:1 to 1:1000 (w/w), or from 1000:1 to 1:100, or from 1000:1 to 1:10, or from 1000:1 to 1:1, or from 1000:1 to 10:1, or from 100:1 to 10:1, or from 90:1 to 10:1, or from 80:1 to 10:1, or from 70:1 to 10:1, or from 60:1 to 10:1, or from 50:1 to 10:1, or from 40:1 to 10:1, or from 30:1 to 10:1 (w/w). The lipid can be present in a ratio to the CDK9 inhibitor of about 50:1 (w/w), or 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, or about 10:1 (w/w).

In some embodiments, the lipid is present in a ratio to the CDK9 inhibitor of from 1000:1 to 1:1 (w/w). In some embodiments, the lipid is present in a ratio to the CDK9 inhibitor of from 100:1 to 10:1 (w/w). In some embodiments, the lipid is present in a ratio to the CDK9 inhibitor of about 50:1 (w/w).

### B. Compositions

The compositions of the present invention can be prepared in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. The compositions of the present invention can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present invention can be administered transdermally. The compositions of this invention can also be administered by intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). Accordingly, the present invention also provides pharmaceutical compositions including a pharmaceutically acceptable carrier or excipient and the compound of the present invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”).

In powders, the carrier is a finely divided solid, which is in a mixture with the finely divided active component. In tablets, the active component is mixed with the carrier having the necessary binding properties in suitable proportions and compacted in the shape and size desired. The powders and tablets preferably contain from 5% or 10% to 70% of the compound the present invention.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants: cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol. Push-fit capsules can contain the compound of the present invention mixed with a filler or binders such as lactose or starches, lubricants such as talc or magnesium stearate, and, optionally, stabilizers. In soft capsules, the compound of the present invention may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the compound of the present invention is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the compound of the present invention in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxy benzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound of the present invention in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compositions of the present invention can also be delivered as microspheres for slow release in the body. For example, microspheres can be formulated for administration via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

In another embodiment, the compositions of the present invention can be formulated for parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the formulations of the compositions of the present invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The compositions of the present invention can be delivered by any suitable means, including oral, parenteral and topical methods. Transdermal administration methods, by a topical route, can be formulated as applicator sticks, solutions, suspensions, emulsions, gels, creams, ointments, pastes, jellies, paints, powders, and aerosols.

The pharmaceutical preparation is preferably in unit dosage form. In such form the preparation is subdivided into unit doses containing appropriate quantities of the compounds of the present invention. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

In some embodiments, the composition further comprises an amino acid and a lipid. Suitable amino acids and lipids for use in the compositions of the present invention are described herein.

### C. Microparticles

The present invention also provides microparticles for treating inflammation. In some embodiments, the present invention provides a microparticle composition comprising a plurality of microparticles, wherein each microparticle comprises: an amino acid; a lipid; and a cyclin-dependent kinase 9 (CDK9) inhibitor.

### Amino Acids and Peptides

Amino acids useful in the compositions and methods of the present invention include, but are not limited to, hydrophobic amino acids, hydrophilic amino acids, and other amino acids. Representative hydrophobic amino acids include, but are not limited to, alanine, valine, isoleucine, leucine, methionine, phenylalanine, tyrosine, tryptohan, and derivatives and analogs thereof. The amino acid can have any suitable stereochemistry. For example, the amino acid can be in the D- or L-configuration.

In some embodiments, the present invention provides a microparticle composition comprising a plurality of microparticles, wherein each microparticle comprises: a hydrophobic amino acid or a hydrophobic peptide, or combinations thereof: a lipid; and a cyclin-dependent kinase 9 (CDK9) inhibitor. In some embodiments, the present invention provides a microparticle composition comprising a plurality of microparticles, wherein each microparticle comprises: a hydrophobic amino acid; a lipid; and a cyclin-dependent kinase 9 (CDK9) inhibitor.

In some embodiments, the hydrophobic amino acid includes alanine, valine, isoleucine, leucine, methionine, phenylalanine, tyrosine, tryptohan, or combinations thereof. In some embodiments, the hydrophobic amino acid includes alanine, valine, isoleucine, leucine, or combinations thereof. In some embodiments, the hydrophobic amino acid includes isoleucine, leucine, or combinations thereof. In some embodiments, the hydrophobic amino acid includes isoleucine. In some embodiments, the hydrophobic amino acid includes L-isoleucine.

In some embodiments, the present invention provides a microparticle composition comprising a plurality of microparticles, wherein each microparticle comprises: L-isoleucine; a lipid; and a cyclin-dependent kinase 9 (CDK9) inhibitor.

Hydrophobic peptides useful in the microparticles of the present invention include peptides having a 2, 3, 4 or more hydrophobic amino acids such as alanine, valine, isoleucine, leucine, methionine, phenylalanine, tyrosine, tryptohan, or combinations thereof. In some embodiments, the present invention provides a microparticle composition comprising a plurality of microparticles, wherein the hydrophobic peptide comprises trileucine.

The Hydrophobic amino acid or hydrophobic peptide can be present in the microparticles in any suitable amount. For example, the Hydrophobic amino acid or hydrophobic peptide can be present in an amount of from 50 to 99% (w/w), or from 55 to 95%, or from 60 to 95%, or from 65 to 95%, or from 70 to 95%, or from 75 to 95%, or from 80 to 95%, or from 85 to 95%, or from 86 to 94%, or from 87 to 93%, or from 88 to 92%, or from 89 to 91% (w/w). The Hydrophobic amino acid or hydrophobic peptide can also be present in the microparticles in an amount of about 50% (w/w), or 55, 60, 65, 70, 75, 80, 81, 82, 83, 84, 85, 86, 87, 88, 89, 90, 91, 92, 93, 94, or about 95% (w/w). The Hydrophobic amino acid or hydrophobic peptide can also be present in the microparticles in an amount of about 89.0% (w/w), or 89.1, 89.2, 89.3, 89.4, 89.5, 89.6, 89.7, 89.8, 89.9, 90.0, 90.1, 90.2, 90.3, 90.4, 90.5, 90.6, 90.7, 90.8, 90.9, or about 91.0% (w/w).

### Lipids

Lipids useful in the compositions and methods of the present invention can include a variety of lipids. Suitable lipids can include but are not limited to fats, fatty acids, waxes, sterols, cholesterol, fat-soluble vitamins. monoglycerides, diglycerides, phospholipids, sphingolipids, glycolipids, derivatized lipids, and the like.

In some embodiments, the lipid is a fatty acid. The fatty acids can be saturated, mono-unsaturated or poly-unsaturated. Examples of fatty acids include, but are not limited to, butyric acid (C4), caproic acid (C6), caprylic acid (C8), capric acid (C10), lauric acid (C12), myristic acid (C14), palmitic acid (C16), palmitoleic acid (C16), stearic acid (C18), isostearic acid (C18), oleic acid (C18), vaccenic acid (C18), linoleic acid (C18), alpha-linoleic acid (C18), gamma-linolenic acid (C18), arachidic acid (C20), gadoleic acid (C20), arachidonic acid (C20), eicosapentaenoic acid (C20), behenic acid (C22), erucic acid (C22), docosahexaenoic acid (C22), lignoceric acid (C24) and hexacosanoic acid (C26).

In some embodiments, the lipid is a phosphocoline lipid. Suitable phospholipids include but are not limited to phosphatidylcholine (PC), phosphatidic acid (PA), phosphatidylethanolamine (PE), phosphatidylglycerol (PG), phosphatidylserine (PS), and phosphatidylinositol (PI). Non-cationic lipids include but are not limited to dimyristoyl phosphatidyl choline (DMPC), distearoyl phosphatidyl choline (DSPC), dioleoyl phosphatidyl choline (DOPC), dipalmitoyl phosphatidyl choline (DPPC), dimyristoyl phosphatidyl glycerol (DMPG), distearoyl phosphatidyl glycerol (DSPG), dioleoyl phosphatidyl glycerol (DOPG), dipalmitoyl phosphatidyl glycerol (DPPG), dimyristoyl phosphatidyl serine (DMPS), distearoyl phosphatidyl serine (DSPS), dioleoyl phosphatidyl serine (DOPS), dipalmitoyl phosphatidyl serine (DPPS), dioleoyl phosphatidyl ethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearoyl-2-oleoyl-phosphatidyethanolamine (SOPE), 1,2-dielaidoyl-sn-glycero-3-phophoethanolamine (transDOPE), and cardiolipin.

In some embodiments, the lipid is 1,2-diheptanoyl-sn-glycero-3-phosphocholine (DHPC), 1,2-dilauroyl-sn-glycero-3-phosphocholine (DLPC), 1,2-Dimyristoyl-sn-glycero-3-phosphocholine (DMPC), 1,2-dipalmitoyl-sn-glycero-3-phosphocholine (DPPC), or 1,2-distearoyl-sn-glycero-3-phosphocholine (DSPC), or combinations thereof. In some embodiments, the lipid is dipalmitoyl phosphatidyl choline (DPPC).

The lipids can also include derivatized lipids, such as PEGylated lipids. PEGylated lipids generally contain a lipid moiety as described herein that is covalently conjugated to one or more PEG chains. The PEG can be linear or branched, wherein branched PEG molecules can have additional PEG molecules emanating from a central core and/or multiple PEG molecules can be grafted to the polymer backbone. PEG can include low or high molecular weight PEG, e.g., PEG500, PEG2000, PEG3400, PEG5000, PEG6000, PEG9000, PEG10000. PEG20000, or PEG50000 wherein the number, e.g., 500, indicates the average molecular weight. Derivatized lipids can include, for example, DSPE-PEG2000, cholesterol-PEG2000, DSPE-polyglycerol, or other derivatives generally well known in the art.

The lipids can be present in the microparticles in any suitable amount. For example, the lipids can be present in an amount of from 1 to 50% (w/w), or 1 to 45%, or 1 to 40%, or 1 to 35%, or 1 to 30%, or 1 to 25%, or 1 to 20%, or 1 to 15%, or 5 to 15%, or 6 to 14%, or 7 to 13%, or 8 to 12%, or 9 to 11% (w/w). The lipids can be present in the microparticles in an amount of about 1% (w/w), or 2, 3, 4, 5, 6, 7, 8, 9, 10, 11, 12, 13, 14, 15, 16, 17, 18, 19, 20, 25, 30, 35, 40, 45, or about 50% (w/w).

The lipids can also be present in any suitable ratio to the L-isoleucine, such as a ratio of the L-isoleucine to the lipid of from 1000:1 to 1:1000 (w/w), or from 1000:1 to 1:100, or from 1000:1 to 1:10, or from 1000:1 to 1:1, or from 100:1 to 1:1, or from 75:1 to 1:1, or from 50:1 to 1:1, or from 40:1 to 1:1, or from 30:1 to 1:1, or from 20:1 to 1:1, or from 15:1 to 5:1, or from 12:1 to 6:1 (w/w). The L-isoleucine can be present in a ratio to the lipid of about 50:1 (w/w), or 45:1, 40:1, 35:1, 30:1, 25:1, 20:1, 15:1, 14:1, 13:1, 12:1, 11:1, 10:1, 9:1, 8:1, 7:1, 6:1, 5:1, or about 1:1 (w/w).

In some embodiments, the L-isoleucine is present in a ratio to the lipid of from 1000:1 to 1:10 (w/w). In some embodiments, the L-isoleucine is present in a ratio to the lipid of from 100:1 to 1:1 (w/w). In some embodiments, the L-isoleucine is present in a ratio to the lipid of about 9:1 (w/w).

### Microparticles

In some embodiments, the microparticle comprises L-isoleucine:DPPC (1,2-dipalmitoyl-sn-glycero-3-phosphocholine); and flavopiridol, wherein the L-isoleucine:DPPC:flavopiridol. In some embodiments, the L-isoleucine:DPPC:flavopiridol ratio is about 89.8:10.0:0.2 (w/w).

The microparticles can be of any suitable size. For example, the microparticles can have a mean geometric diameter of from 0.1 to 100 microns, or from 0.1 to 50, or from 0.1 to 25, or from 0.1 to 10, or from 1 to 10, or from 1 to 7.5, or from 1 to 5, or from 1 to 2.5, or from 3 to 7 microns. The microparticles can have a mean geometric diameter of about 1 micron, or 1.5, 2, 2.5, 3, 3.5, 4, 4.5, 5, 5.5, 6, 6.5, 7, 7.5, 8, 8.5, 9, 9.5, or about 10 microns. In some embodiments, the microparticle has a mean geometric diameter of from 1 to 10 microns.

In some embodiments, the microparticles of the present invention do not include one or more components such as sugar or an inorganic salt. The microparticles of the present invention exclude one or more sugars such as, but not limited to, glucose, sucrose, fructose, galactose, mannose, ribose, lactose, trehalose, mannitol, sorbitol, and xylitol. The microparticles of the present invention exclude one or more inorganic salts such as, but not limited to, sodium chloride (NaCl), potassium chloride (KCl), calcium chloride (CaCl₂)), and magnesium chloride (MgCl₂).

In some embodiments, the microparticle does not include CaCl₂). In some embodiments, the microparticle does not include glucose. In some embodiments, the microparticle does not include CaCl₂) or glucose. In some embodiments, the microparticle does not include CaCl₂) and glucose.

In some embodiments, the microparticle consists essentially of L-isoleucine; DPPC (1,2-dipalmitoyl-sn-glycero-3-phosphocholine); and flavopiridol, wherein the L-isoleucine:DPPC:flavopiridol, wherein the L-isoleucine:DPPC:flavopiridol ratio is about 89.8:10.0:0.2 (w/w).

In some embodiments, the microparticle consists of L-isoleucine:DPPC (1,2-dipalmitoyl-sn-glycero-3-phosphocholine); and flavopiridol, wherein the L-isoleucine:DPPC:flavopiridol, wherein the L-isoleucine:DPPC:flavopiridol ratio is about 89.8:10.0:0.2 (w/w).

In some embodiments, the microparticles release the CDK9 inhibitor at approximately a constant rate over the treatment period. In some embodiments, the microparticles release at a constant rate after an initial release of about 3% to about 10% of the encapsulated CDK9 inhibitor. In some embodiments, the initial release occurs within about 24 hours. In some embodiments, the initial release occurs within about 12 hours. In some embodiments, the initial release occurs within about 8 hours. In some embodiments, the initial release occurs within about 1 hour. In some embodiments, the microparticle releases from about 3% to about 30%, about 3% to about 20%, about 3% to about 10%, about 5% to about 30%, about 5% to about 20%, or from about 5% to about 10% of the CDK9 inhibitor over 24 hours. In some embodiments, the microparticle releases from about 5% to about 40%, about 5% to about 30%, about 5% to about 20%, about 10% to about 40%, about 10% to about 30%, about 10% to about 20%, or from about 10% to about 15% of the CDK9 inhibitor over 2 days. In some embodiments, the microparticle releases from about 10% to about 50%, about 10% to about 40%, about 10% to about 30%, about 15% to about 40%, about 15% to about 30%, or from about 15% to about 25% of the CDK9 inhibitor over 5 days. In some embodiments, the microparticle releases from about 20% to about 70%, about 20% to about 60%, about 20% to about 50%, about 20% to about 40%, or from about 25% to about 35% of the CDK9 inhibitor over 8 days. In some embodiments, the microparticle releases from about 30% to about 70%, about 30% to about 60%, about 30% to about 50%, about 40% to about 70%, about 40% to about 60%, or from about 40% to about 50% of the CDK9 inhibitor over 12 days. In some embodiments, the microparticle releases from about 40% to about 80%, about 40% to about 70%, about 50% to about 70%, or from about 55% to about 65% of the CDK9 inhibitor over 15 days. In some embodiments, the microparticle releases from about 40% to about 80%, about 50% to about 80%, about 60% to about 80%, or from about 65% to about 75% of the CDK9 inhibitor over 19 days. In some embodiments, the microparticle releases from about 40% to about 90%, about 50% to about 90%, about 60% to about 90%, about 70% to about 90%, or from about 75% to about 85% of the CDK9 inhibitor over 22 days. In some embodiments, the microparticle releases from about 50% to about 90%, about 60% to about 90%, about 70% to about 90%, or from about 80% to about 90% of the CDK9 inhibitor over 26 days. In some embodiments, the microparticle releases from about 60% to about 95%, about 70% to about 95%, about 80% to about 95%, or from about 85% to about 95% of the CDK9 inhibitor over 30 days.

In some embodiments, at least about 80% of the encapsulated CDK9 inhibitor is released by the end of the treatment period. In some embodiments, the amount of encapsulated CDK9 inhibitor released is at least about 85%, at least about 90%, at least about 94%, at least about 95%, at least about 96%, at least about 97%, at least about 98%, at least about 99%, or at least about 99.5%. In some embodiments, the treatment period is at least about 24 hours, at least about 2 days, at least about 5 days, at least about 7 days, at least about 10 days, at least about 14 days, at least about 20 days, at least about 21 days, at least about 28 days, at least about 30 days, at least about 31 days, at least about 40 days, at least about 42 days, at least about 45 days, at least about 48 days, at least about 50 days, or at least about 60 days. In some embodiments, the treatment period is less than about 60 days, less than about 55 days, less than about 50 days, less than about 45 days, less than about 40 days, less than about 30 days, less than about 28 days, less than about 25 days, less than about 21 days, less than about 20 days, less than about 14 days, less than about 10 days, less than about 7 days, less than about 5 days, or less than about 2 days.

In some embodiments, the microparticle releases from about 3% to about 10% of the CDK9 inhibitor over about 24 hours; from about 10% to about 20% of the CDK9 inhibitor over about 2 days; from about 15% to about 25% of the CDK9 inhibitor over about 5 days; from about 25% to about 35% of the CDK9 inhibitor over about 8 days; from about 40% to about 50% of the CDK9 inhibitor over about 12 days; from about 55% to about 65% of the CDK9 inhibitor over about 15 days; from about 65% to about 75% of the CDK9 inhibitor over about 19 days; from about 75% to about 85% of the CDK9 inhibitor over about 22 days; from about 80% to about 90% of the CDK9 inhibitor over about 26 days; and/or from about 85% to about 95% of the CDK9 inhibitor over about 30 days.

In some embodiments, the microparticle releases the CDK9 inhibitor over a duration selected from the group consisting of about 24 hours, about 2 days, about 5 days, about 10 days, about 14 days, about 21 days, about 30 days, about 45 days, and about 60 days. In some embodiments, the microparticle releases from about 5% to about 40%, about 5% to about 30%, about 5% to about 20%, about 10% to about 40%, about 10% to about 30%, about 10% to about 20%, or from about 10% to about 15% of the CDK9 inhibitor over 2 days following administration. In some embodiments, the microparticle releases from about 10% to about 50%, about 10% to about 40%, about 10% to about 30%, about 15% to about 40%, about 15% to about 30%, or from about 15% to about 25% of the CDK9 inhibitor over 5 days following administration. In some embodiments, the microparticle releases from about 20% to about 70%, about 20% to about 60%, about 20% to about 50%, about 20% to about 40) %, or from about 25% to about 35% of the CDK9 inhibitor over 8 days following administration. In some embodiments, the microparticle releases from about 30% to about 70%, about 30% to about 60%, about 30% to about 50%, about 40% to about 70%, about 40% to about 60%, or from about 40% to about 50% of the CDK9 inhibitor over 12 days following administration. In some embodiments, the microparticle releases from about 40% to about 80%, about 40% to about 70%, about 50% to about 70%, or from about 55% to about 65% of the CDK9 inhibitor over 15 days following administration. In some embodiments, the microparticle releases from about 40% to about 80%, about 50% to about 80%, about 60% to about 80%, or from about 65% to about 75% of the CDK9 inhibitor over 19 days following administration. In some embodiments, the microparticle releases from about 40% to about 90%, about 50% to about 90%, about 60% to about 90%, about 70% to about 90%, or from about 75% to about 85% of the CDK9 inhibitor over 22 days following administration. In some embodiments, the microparticle releases from about 50% to about 90%, about 60% to about 90%, about 70% to about 90%, or from about 80% to about 90% of the CDK9 inhibitor over 26 days following administration. In some embodiments, the microparticle releases from about 60% to about 95%, about 70% to about 95%, about 80% to about 95%, or from about 85% to about 95% of the CDK9 inhibitor over 30 days following administration.

Pharmaceutical compositions of the invention comprise microparticles of the invention dispersed or suspended in a pharmaceutically acceptable carrier. As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, dyes, like materials and combinations thereof, as would be known to one of ordinary skill in the art (see, for example. Remington's Pharmaceutical Sciences, 18th Ed. Mack Printing Company, 1990, pp. 1289-1329, incorporated herein by reference). Except insofar as any conventional carrier is incompatible with the microparticles of the invention, its use in the pharmaceutical compositions is contemplated. It is anticipated that the compositions of the invention will be administered primarily by injection or other parenteral methods: however, gel and aerosol compositions may also be used, for example, for application during a surgical procedure. Suitable carriers include water, water for injection, saline, phosphate buffered saline, and the like. Compositions of the invention can further include propellants, anti-aggregation agents, and the additional agents listed above.

In some embodiments, the present invention provides a composition for use in treating inflammation or respiratory fibrosis in a subject in need thereof, wherein the composition comprises a cyclin-dependent kinase 9 (CDK9) inhibitor. The composition for treating inflammation or respiratory fibrosis can be any composition of the present invention. In some embodiments, the present invention provides a composition for use in treating respiratory fibrosis in a subject in need thereof, wherein the composition comprises a cyclin-dependent kinase 9 (CDK9) inhibitor.

### Methods of Preparing Microparticles

The present invention also provides methods of preparing the microparticles. In some embodiments, the present invention provides a method of preparing a plurality of particles of the present invention, comprising
sonicating a first reaction mixture comprising L-isoleucine and 1,2-dipalmitoyl-sn-glycero-3-phosphocholine (DPPC), water, and ethanol, wherein the L-isoleucine and DPPC are present in a ratio of about 9:1 (w/w), and wherein the ethanol:water ratio is about 70:30 (v/v), to prepare a 0.3% (w/v) feed mixture:
applying the feed mixture to a microfluidic piezo array to form a spray of micronized droplets; and
drying the droplets to afford the plurality of particles.

The microfluidic piezo array can be actuated at any suitable frequency and power. Representative frequencies can be from 0.1 kHz to 1000 kHz, or from 1 kHz to 1000 kHz, from 10 kHz to 1000 kHz, from 10 kHz to 500 kHz, from 10 kHz to 250 kHz, from 10 kHz to 200 kHz, from 50 kHz to 150 kHz, or from 75 kHz to 125 kHz. For example, the frequency can be about 10 KHz, or 20, 30, 40, 50, 60, 70, 80, 90, 100, 110, 120, 130, 140, 150, 160, 170, 180, 190, or about 200 kHz. The frequency can also be about 110 kHz, or 111, 112, 113, 114, 115, 116, 117, 118, 119, or about 120 KHz.

Representative powers can be from 0.1 to 1000 V, or from 1 to 900 V, from 1 to 500 V, from 1 to 250 V, from 1 to 100 V, 1 to 75 V, 10 to 50 V, 15 to 45 V, 20 to 40 V, or from 25 to 35 V. For example, the power can be about 1 V, or 5, 10, 15, 20, 25, 30, 35, 40, 45, 50, 55, 60, 65, 70, 75, 80, 85, 90, 95, or about 100 V.

In some embodiments, the microfluidic piezo array is actuated at 113 kHz with 30V of power.

### D. Liquid Compositions

The present invention also provides liquid compositions. In some embodiments, the present invention provides a liquid composition comprising: a citric acid buffer having a pH of from 2 to 7; and a cyclin-dependent kinase 9 (CDK9) inhibitor.

The citric acid buffer can include any suitable components. For example, the citric acid buffer can include one or more of citric acid, citric acid monohydrate, and trisodium citrate dihydrate. The citric acid buffer can have any suitable pH from 2 to 7. For example, the citric acid buffer can have a pH of from 3 to 6, or from 4 to 5. The citric acid buffer can have a pH of about 2, 3, 4, 5, 6 or 7. In some embodiments, the citric acid buffer comprises citric acid monohydrate and trisodium citrate dihydrate, having a pH of from 4 to 5. In some embodiments, the citric acid buffer comprises citric acid monohydrate and trisodium citrate dihydrate, having a pH of from 4 to 5.

In some embodiments, the CDK9 inhibitor is flavopiridol, SNS-032, voruciclib, or a derivative thereof, or pharmaceutically acceptable salt thereof. In some embodiments, the CDK9 inhibitor is flavopiridol, SNS-032, or voruciclib, or a pharmaceutically acceptable salt thereof. In some embodiments, the CDK9 inhibitor is flavopiridol.

The liquid composition can include a variety of other components, such as, but not limited to, inorganic salts. Representative inorganic salts include, but are not limited to, sodium chloride, potassium chloride, magnesium chloride, and calcium chloride. In some embodiments, the liquid composition also includes sodium chloride.

In some embodiments, the liquid composition comprises: the citric acid buffer, having a pH of from 4 to 5; flavopiridol at a concentration of about 120 μM; and sodium chloride at a concentration of about 70 mM.

### IV. Methods of Administrating

The present invention provides methods of administering the microparticles and liquid compositions of the present invention. In some embodiments, the present invention provides a method of administering a therapeutically effective amount of a CDK9 inhibitor to a subject in need thereof, comprising administering to the subject a microparticle composition of the present invention, or a liquid composition of the present invention, via respiratory administration.

Suitable dosage ranges for the microparticles and liquid compositions of the present invention include from about 0.1 mg to about 100 mg, or about 1 mg to about 1000 mg, or about 1 to about 100 mg, or about 1 to about 50 mg, or about 1 to about 25 mg, or about 1 to about 10 mg, or about 10 mg to about 750 mg, or about 25 mg to about 500 mg, or about 50 mg to about 250 mg. Suitable dosages for the compound of the present invention include about 1 mg, or about 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900 or about 1000 mg.

The microparticles and liquid compositions of the present invention can be administered at any suitable frequency, interval and duration. For example, the microparticles and liquid compositions of the present invention can be administered once an hour, or two, three or more times an hour, once a day, or two, three, or more times per day, or once every 2, 3, 4, 5, 6, or 7 days, so as to provide the preferred dosage level. When the microparticles and liquid compositions of the present invention is administered more than once a day, representative intervals include 5, 10, 15, 20, 30, 45 and 60 minutes, as well as 1, 2, 4, 6, 8, 10, 12, 16, 20, and 24 hours. The microparticles and liquid compositions of the present invention can be administered once, twice, or three or more times, for an hour, for 1 to 6 hours, for 1 to 12 hours, for 1 to 24 hours, for 6 to 12 hours, for 12 to 24 hours, for a single day, for 1 to 7 days, for a single week, for 1 to 4 weeks, for a month, for 1 to 12 months, for a year or more, or even indefinitely.

### V. Methods of Treating

Another embodiment of the invention is a method of treating a subject in need thereof, comprising administering a therapeutically effective amount of a plurality of microparticles.

In some embodiments, the present invention provides a method of treating a disease or disorder in a subject in need thereof, comprising administering a therapeutically effective amount of a microparticle composition of the present invention, or a liquid composition of the present invention, to the subject via respiratory administration, thereby treating the inflammation.

In some embodiments, the disease or disorder is respiratory inflammation, acute pulmonary inflammation, chronic pulmonary inflammation, acute respiratory distress syndrome (ARDS), chronic pulmonary obstructive disease (COPD), or toxin exposure.

The following clinical end points are non-limiting examples of treatment: reduction in inflammation, reduction in rate of decline of Forced Vital Capacity (FVC), wherein FVC is the total amount of air exhaled during the lung function test, absolute and relative increases from baseline in FVC, absolute increase from baseline in FVC (% Predicted), increase in progression-free survival time, decrease from baseline in St George's Respiratory Questionnaire (SGRQ) total score, wherein SGRQ is a health-related quality of life questionnaire divided into 3 components: symptoms, activity and impact and the total score (summed weights) can range from 0 to 100 with a lower score denoting a better health status, and relative decrease from baseline in high resolution computerized tomography (HRCT) quantitative lung fibrosis (QLF) score, wherein the QLF score ranges from 0 to 100% and greater values represent a greater amount of lung fibrosis and are considered a worse health status.

In some embodiments, the present invention provides a method of treating inflammation in a subject in need thereof, comprising administering a therapeutically effective amount of a microparticle composition of the present invention, or a liquid composition of the present invention, to the subject via respiratory administration, thereby treating the inflammation.

In some embodiments, the inflammation is respiratory inflammation.

In some embodiments, the respiratory administration is inhalation administration or nasal administration.

The subject that can be treated with a method of the present disclosure is a human, or a non-human mammal, for example a companion animal, such as a dog, cat, rat, or the like, or a farm animal, such as a horse, donkey, mule, goat, sheep, pig, or cow, or the like.

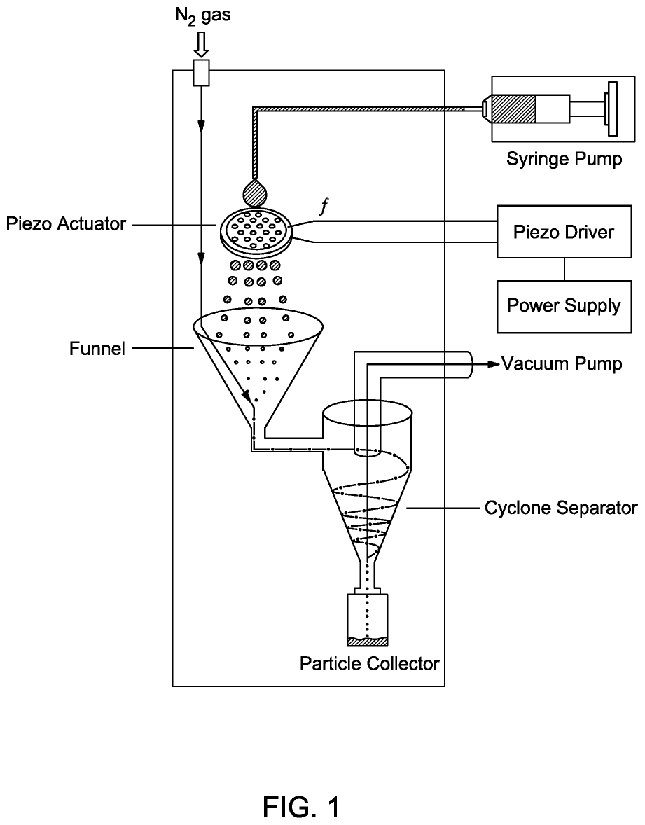

CDK9 inhibitors exert effects on the inflammatory response pathway. For example, the pharmacological CDK9 inhibitor flavopiridol effectively suppresses the activation of a broad range of primary inflammatory response genes, in human cell culture treated with IL-1β for 5 hours (see FIG. 1). Among the 67 different genes (out of 84 total NFκB target genes tested) that were induced by IL-1β, 59 were repressed by flavopiridol co-treatment (including the most-characterized pro-inflammatory cytokines such as IL-1β, Il-6, and TNF). The average magnitude of repression is >86% of maximum induction. These data demonstrate that CDK9 inhibition is highly efficient in suppressing the induction of a broad range of primary inflammatory genes. Importantly, house-keeping genes and non-inducible genes are not affected by CDK9 inhibition short term, indicating potential reduction in side effects.

Current anti-inflammatory drugs either target various components of the upstream inflammatory signaling pathways, or the downstream effector genes (IL-1 antagonists, TNF antagonists, anti-oxidants, etc.). The focus has been on inhibition of the specific pathway(s) so that transcription of corresponding response genes does not occur, or on inhibition of individual downstream effector gene functions. None of these existing investigations have addressed the rate-limiting process of transcriptional elongation that is controlled by CDK9. These existing drugs may be less effective in handling the diverse physiological pro-inflammatory challenges, and may not be able to prevent activation of a broad range of different downstream inflammatory response genes. Therefore, targeting CDK9 that controls the rate-limiting step for all inflammatory gene activation is more effective and efficient. Inhibition of the transcriptional elongation by CDK9 is limited to the primary response inflammatory genes, and CDK9 inhibition does not affect transcription of housekeeping genes and non-inducible genes within the acute inflammatory phase tested, and therefore is not detrimental to cells or tissues in the short term. One advantage of CDK9 inhibition is that it reduces transcriptional elongation of inflammatory genes from numerous inflammatory stimuli. CDK9 can be specifically and reversibly inhibited with small-molecule drugs such as flavopiridol and others disclosed herein, including SNS-032, voruciclib, and flavopiridol. In conjunction with the formulations and methods herein, CDK9 inhibitors are delivered locally to a site of inflammation and thereby reduce, alleviate, prevent or reduce inflammatory response and symptoms thereof.

In some embodiments, the formulated CDK9 inhibitor is administered for such pre-existing condition on a chronic basis, such as once or twice a day, every week, every 2 weeks, every 3 weeks, every month (e.g. 4 weeks), every 5, 6, 7, 8, 9 or 10 weeks. In some embodiments, the formulated CDK9 inhibitor is administered for such pre-existing condition on a chronic basis, until the symptoms, inflammation or other signs of the condition or disease are reduced, ameliorated, dampened or otherwise effected by the treatment. In some embodiments, the formulated CDK9 inhibitor is administered for such pre-existing condition on a chronic basis for the life-time of a subject or from the time of diagnosis or flare-up of the disease or condition.

In some embodiments, the present invention provides a method of treating fibrosis in a subject in need thereof, comprising administering a therapeutically effective amount of a microparticle composition of the present invention, or a liquid composition of the present invention, to the subject via respiratory administration, thereby treating the fibrosis.

The fibrosis that can be treated by the methods of the present invention includes, but is not limited to, respiratory fibrosis, lung fibrosis, idiopathic pulmonary fibrosis, bleomycin-induced pulmonary fibrosis, fibroblastic lesions, activated fibroblast proliferation, inflammation, or myofibroblast genesis. In some embodiments, the fibrosis is respiratory fibrosis.

Additional types of fibrosis can also be treated by the compositions and methods of the present invention including, but not limited to, kidney fibrosis, liver, skin fibrosis, fibroblastic lesions, activated fibroblast proliferation, inflammation, myofibroblast genesis, and non-idiopathic forms of fibrosis. Still other types of fibrosis that can be treated using the compositions and methods of the present invention include lung scarring, progressive pulmonary fibrosis, or fibrosis caused by medication, radiation treatments to the chest, smoking, or an environmental or occupational exposure known to cause pulmonary fibrosis. Pulmonary fibrosis casued by autoimmune disorders, such as rheumatoid arthritis, scleroderma, Sjogren's syndrome, and others, viral infections, and gastroesophageal reflux disease (GERD), and familial pulmonary fibrosis, can be treated by the compositions and methods of the present invention. Exposure to environmental hazards, such as asbestos or silica, or bird or animal droppings, can also cause pulmonary fibrosis that can be treatedy by the compositions and methods of the present invention.

In some embodiments, the present invention provides a method of treating respiratory fibrosis in a subject in need thereof, comprising administering a therapeutically effective amount of a microparticle composition of the present invention, or a liquid composition of the present invention, to the subject via respiratory administration, thereby treating the respiratory fibrosis.

When the disease or disorder is respiratory fibrosis, the following clinical end points are non-limiting examples of treatment: reduction in fibrotic tissue, reduction in inflammation, reduction in fibroblastic lesions, reduction in activated fibroblast proliferation, reduction in myofibroblast genesis, reduction in rate of decline of Forced Vital Capacity (FVC), wherein FVC is the total amount of air exhaled during the lung function test, absolute and relative increases from baseline in FVC, absolute increase from baseline in FVC (% Predicted), increase in progression-free survival time, decrease from baseline in St George's Respiratory Questionnaire (SGRQ) total score, wherein SGRQ is a health-related quality of life questionnaire divided into 3 components: symptoms, activity and impact and the total score (summed weights) can range from 0 to 100 with a lower score denoting a better health status, and relative decrease from baseline in high resolution computerized tomography (HRCT) quantitative lung fibrosis (QLF) score, wherein the QLF score ranges from 0 to 100% and greater values represent a greater amount of lung fibrosis and are considered a worse health status. Non-limiting examples clinical end points for fibrosis treatment and tests that can be performed to measure said clinical end points are described in the following clinical trials: NCT03733444 (clinicaltrials.gov/ct2/show/NCT03733444) (last accessed on Jan. 9, 2019). NCT00287729 (clinicaltrials.gov/ct2/show/NCT00287729) (last accessed on Jan. 9, 2019), NCT00287716 (clinicaltrials.gov/ct2/show/NCT00287716) (last accessed on Jan. 9, 2019), NCT02503657 (clinicaltrials.gov/ct2/show/NCT02503657) (last accessed on Jan. 9, 2019). NCT00047645 (clinicaltrials.gov/ct2/show/NCT00047645) (last accessed on Jan. 9, 2019), NCT02802345 (clinicaltrials.gov/ct2/show/NCT02802345) (last accessed on Jan. 9, 2019), NCTO 1979952 (clinicaltrials.gov/ct2/show/NCT01979952) (last accessed on Jan. 9, 2019), NCT00650091 (clinicaltrials.gov/ct2/show/NCT00650091) (last accessed on Jan. 9, 2019). NCT01335464 (clinicaltrials.gov/ct2/show/NCT01335464) (last accessed on Jan. 9, 2019), NCT01335477 (clinicaltrials.gov/ct2/show/NCT01335477) (last accessed on Jan. 9, 2019), NCT01366209 (clinicaltrials.gov/ct2/show/NCT01366209) (last accessed on Jan. 9, 2019). Further non-limiting examples clinical endpoints for fibrosis treatment and tests that can be performed to measure said clinical end points are described in King et al, (2014) N Engl J Med. May 29; 370(22):2083-92 and Richeldi et al, (2014) N Engl J Med. May 29; 370(22):2071-82.

In some embodiments, the respiratory fibrosis is lung fibrosis.

In some embodiments, the respiratory administration is inhalation administration or nasal administration.

In some embodiments, the subject is a human.

In some embodiments, the method includes administering an anti-fibrotic agent. The methods of the present invention can include administering an anti-fibrotic agent or drug such as, but not limited to, pirfenidone, idebenone, nintedanib, Ifenprodil, n-acetyl cysteine, penetaxin. TD139 and corticosteroids. Additional agents useful as anti-fibrotic agents or drugs include one or more of colchicine, D-penicillamine, pirfenidone (5-methyl-1-phenyl-2-[1H]-pyridone), interferon-β1a, relaxin, lovastatin, beractant, N-acetylcysteine, keratinocyte growth factor, captopril, hepatocyte growth factor, Rhokinase inhibitor, thrombomodulin-like protein, bilirubin, PPARγ (peroxisome proliferator-activated receptor gamma) activator, imatinib, and interferon-γ. Additional agents are known in the literature, e.g., JP A No. 8-268906, WO 00/57913, JP A No. 2002-371006, JP A No. 2003-119138, JP A No. 2005-513031, JP A No. 2005-531628, JP A No. 2006-502153, WO 2006/068232, and Ann Intern Med. 2001; 134(2): 136-51. In some embodiments, the anti-fibrotic agent is pirfenidone, idebenone, nintedanib, Ifenprodil, n-acetyl cysteine, penetaxin, TD139, a corticosteroid, colchicine, D-penicillamine, pirfenidone (5-methyl-1-phenyl-2-[1H]-pyridone), interferon-β1a, relaxin, lovastatin, beractant, N-acetylcysteine, keratinocyte growth factor, captopril, hepatocyte growth factor, Rhokinase inhibitor, thrombomodulin-like protein, bilirubin, PPARγ (peroxisome proliferator-activated receptor gamma) activator, imatinib, or interferon-γ.

In some embodiments, the present invention provides a method of treating inflammation or respiratory fibrosis in a subject in need thereof, comprising administering a therapeutically effective amount of a composition comprising a cyclin-dependent kinase 9 (CDK9) inhibitor. The compositions useful in the method of the present invention include any composition of the present invention. In some embodiments, the present invention provides a method of treating respiratory fibrosis in a subject in need thereof, comprising administering a therapeutically effective amount of a composition comprising a cyclin-dependent kinase 9 (CDK9) inhibitor.

### VI. Examples

### Materials

1,2-dipalmitoyl-sn-glycero-3-phosphocholine (DPPC) was purchased from Avanti Polar Lipids (Birmingham. AL). Flavopiridol was purchased from Cayman Chemical Company (Ann Arbor, MI). L-leucine. L-isoleucine, D-(+)-Glucose, Calcium chloride (CaCl₂)), Tween-20, and Ethanol 100% was purchased from Sigma Aldrich (St. Louis, MO). Phosphate-buffered saline (PBS) (1×) was purchased from Mediatech (Manassas, VA). Gaseous nitrogen was purchased from Praxair (Danbury. CT). Poly(tetrafluoroethylene) (PTFE) tubing was purchased from Cole-Parmer (Vernon Hills. IL). 96 well plates, 10- and 20-mL glass scintillation vials, and 1.00 mm ID glass capillary tubes were purchased from Fisher Scientific (Hampton, NH). Variable area flowmeter was purchased from Dwyer Instruments Inc. (Michigan City. IN) Dulbecco's modified Eagle's Medium (DMEM), Fetal Bovine Serum (FBS), and 2× Lysis reagent were purchased from Invitrogen (Waltham, MA). Human embryonic kidney 293 cells (HEK-293) were purchased from ATCC (Manassas, VA). Nuclear factor kappa B (NF-κB) reporter (cat #H-60650) and One-Step Luciferase Assay System were purchased from BPS Bioscience (San Diego, CA). Tumor necrosis factor alpha (TNF-α) was purchased from Peprotech (Cranbury, NJ). Ultrapure water with a resistivity of 18.2 MΩ·cm was generated using a Millipore Milli-Q system (EMD Millipore, Billerica, MA). Gaseous nitrogen and gaseous CO₂ were purchased from Praxair (Danbury, CT).

### Example 1. Production of Flavopiridol Loaded L-isoleucine/DPPC Microparticles

The Microfluidic Piezo and Cyclone Apparatus (MPCA) was designed and fabricated and is shown schematically in FIG. 1. The MPCA was designed and fabricated using components for spray drying technique and further includes a piezoelectric nebulizer disk with orifice array and cyclone design. The piezo actuated orifice array is a commercial humidifier (Steiner & Martins, Inc.), composed of a stainless-steel disk with an array of 4-11 μm holes, ceramic piezo rings are fixed on the top and bottom of the array. The cyclone separator with funnel was designed in Solidworks® CAD software with a cylinder-on-cone design with a tangential inlet and manufactured in PTFE.

Feed solutions of L-isoleucine/DPPC (ILD) were prepared using similar methods previously reported (Eedara et al. (2018) International Journal of Pharmaceutics 542 (1-2), 72-81). Briefly, 27 mg of L-isoleucine was added to a 10 mL glass scintillation containing 3 mL of Milli-Q water. 3 mg of DPPC (1,2-dipalmitoyl-sn-glycero-3-phosphocholine (Avanti Polar Lipids)) was added to a 10 mL glass scintillation vial containing 7 mL of ethanol. Both vials were closed tightly and sonicated for 15 minutes until complete dissolution. Following sonication, the entire contents of the DPPC vial was added to the L-isoleucine vial, and sonicated for 15 minutes, making a 0.3% (w/v) ILD feed solution.

The ILD feed solution was pulled into a 10 ml syringe and equipped on a syringe pump of the Microfluidic Piezo and Cyclone Apparatus (MPCA) shown in FIG. 1. The ILD feed solution was pumped into PTFE tubing via syringe pump at 22 mL/hr. The end of the PTFE tubing contained a 30 AWG blunt needle hovering 1 cm directly over the piezo actuated orifice array. The ILD solution was dropped onto the microfluidic piezo array which was actuated via custom piezo driver at 113 kHz with 30V of power. The gravitational force caused the large droplet on top of the device to be pulled into the array, causing the formation of a spray of micronized droplets. At the top of the acrylic box, which houses the entire apparatus, a nitrogen inlet fed into the enclosure at 15 L/min. The nitrogen gas carried and began to dry the micronized droplets for 55 cm until it reached the custom PTFE funnel where the vacuum pull led to the cyclone chamber. In the cyclone chamber, the particles strike the side walls causing a decrease in momentum effectively landing in the screwed on 20 mL glass scintillation collection vial, while the gas and evaporated solvent traveled to the vacuum outlet and are removed via an RV5 vacuum pump (Edwards Vacuum). The synthesis was performed at ambient room temperature, 24° C. Synthesis parameters are provided in Table 1.

TABLE 1
MPCA operation parameters for particle synthesis

| Parameter | Value | |
|---|---|---|
| Micro-piezo orifice diameter | 11 | μm |
| Feed solution temperature | 23 ± 1° | C. |
| Feed solution flow rate | 22.0 | mL/hr |
| N2 gas flow rate | 15 | L/min |
| N2 gas temperature | 23 ± 1° | C. |
| Piezoelectric driving frequency | 113 | kHz |

To demonstrate robustness, 0.1 and 0.5% (w/v) total excipient concentrations were also prepared using identical conditions. Flavopiridol incorporation into the formulation (ILDF: L-IsoLeucine, DPPC, Flavopiridol) was prepared by adding 28.8 μL of a 1 mg/ml Flavopiridol (Cayman Chemical Company) in ethanol solution, into the DPPC vial described above.

### Example 2. Measurements of Loading Capacity and Efficiency of Flavopiridol in L-Isoleucine/DPPC Microparticles

Flavopiridol loading in the ILDF (L-IsoLeucine, DPPC, Flavopiridol) formulation microparticles was determined by dispersing ˜4.8 mg of particles (mₚ) in 1 mL of 70% (v/v) ethanol in water. The dispersion was sonicated for 15 minutes, until complete dissolution of the particles was complete. The dissolved solution was loaded in a 1 mL syringe and filtered into a quartz cuvette using a 0.2 μm PTFE syringe filter (Sigma Aldrich). The filtered solution was subject to UV-Vis spectroscopy using a Denovix DS-11 spectrometer, and the absorbance (A) at 358 nm, characteristic of flavopiridol was measured. The molar extinction coefficient (ϵ) of flavopiridol at 358 nm was determined using a standard concentration range from 3-100 μM, and the concentration of flavopiridol (c) was calculated using Beer's law. The encapsulated mass of flavopiridol (m_{f}) in the ILDF particles was determined by multiplying c with the volume (1.00 mL) and the molar mass of flavopiridol. The loading capacity (LC) of flavopiridol was quantified using eq. (1),

$$LC = ( \frac{{m}_{f}}{{m}_{p}} ) \times 100 \% \qquad ( 1 )$$

The encapsulation efficiency (EE) of flavopiridol was quantified using eq. (2) by dividing the LC with the desired loading capacity of flavopiridol in the formulation (LC_{desired}=0.192% (w/w)).

$$EE = \frac{LC}{{LC}_{desired}} \times 100 \% \qquad ( 2 )$$

Using the MPCA with the parameters listed in Table 1, flavopiridol loaded ILDF particles were produced with the formulation in Table 2.

TABLE 2
Formulations for particle synthesis using MPCA

| | | | | | Total |
|---|---|---|---|---|---|
| | EtOH:Water | IL | DPPC | Flavopiridol | concentration |
| Sample | (% (v/v)) | (%) | (%) | (%) | (% (w/v)) |
| ILD-3 | 70 | 90 | 10 | 0 | 0.3 |
| ILDF | 70 | 89.808 | 10 | 0.192 | 0.3 |

The flavopiridol loading was 0.19% as determined using UV-Vis spectrum. As a negative control, the unloaded ILD particles did not exhibit absorption at λₘₐₓ=358 nm. The Encapsulation Efficiency (EE) in this synthesis was quantified using eq (2) and reached 99±2%.

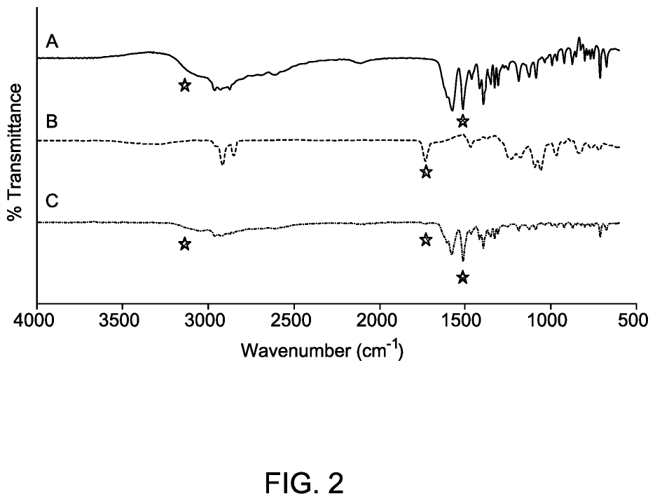

The composition was confirmed using FTIR spectroscopy as shown in FIG. 2. Briefly, <1 mg of solid L-isoleucine, DPPC, and flavopiridol starting materials and produced ILDF formulation particles were placed and compressed on the ATR diamond crystal. The FTIR spectrum was scanned 30 times with 4 cm-1 resolution over the wavenumber range of 4000-400 cm 1 using a Bruker Tensor 27 FTIR (Bruker Corporation). The spectrum of the ILDF formulation (C) most similarly resembles L-isoleucine (A) with a characteristic 1514 cm-1 (star), with a slight presence of the characteristic C═O stretching (star) of DPPC (B) indicated above. Based on the spectrum, L-isoleucine is the major component of the formulation, while DPPC plays a more minor role in the composition.

### Example 3. Scanning Electron Microscopy Imaging of Produced Particles

The microparticle geometry and size were determined using a field emission scanning electron microscope (SEM) (S-4100T, Hitachi High Technologies America). Produced particles stuck to double sided carbon tape mounted on an Al stub were coated with 10 nm of gold using a sputter coater (Ted Pella Inc.) and then transported to the SEM vacuum chamber. The typical acceleration voltage and emission current were 2 kV and 10 μA, respectively. The SEM images were loaded into Image J, where the size of the microparticles was determined using the geometrical Feret diameter (d_{g}). The aerodynamic diameter (dₐ) was calculated using eq. (3), where The tapped density was measured using established protocols¹², where the measured tapped density (σ) which was determined over 1000 taps, and the reference density (φ is 1 mg/mL.

$${d}_{a} = {d}_{g} \sqrt{\frac{σ}{ρ}} \qquad ( 3 )$$

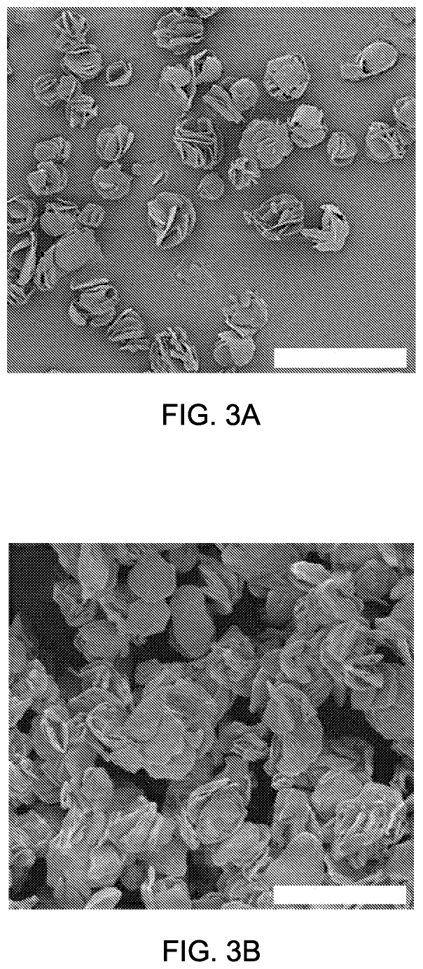

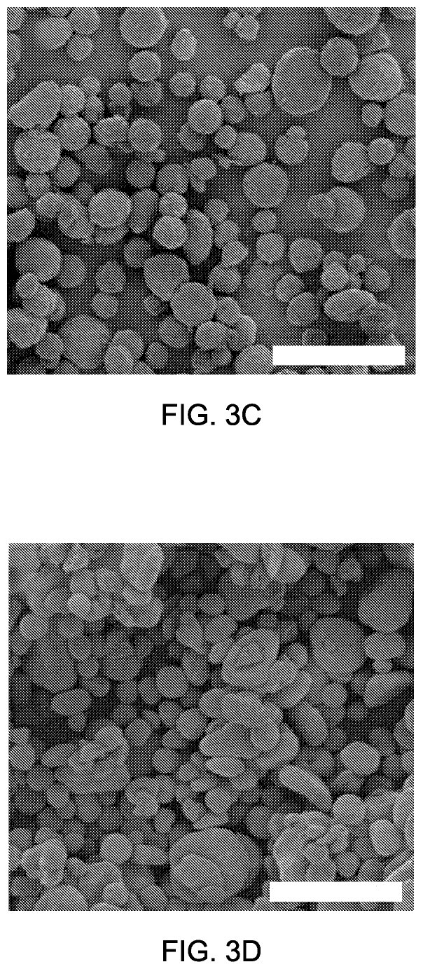

The SEM images are shown in FIG. 3. FIG. 3A shows the morphology of particles with the (90:10) L-isoleucine/DPPC. The addition of a phospholipid to the L-isoleucine formulation resulted in particle morphology composed of outward folded sheets that were analogous to a “rose-bud” morphology. FIG. 3B ((89.25:10:0.75) L-isoleucine/DPPC/CaCl₂)) shows the addition of CaCl₂) to the formulation resulted in heterogeneity in shape with a flattened heterogenous morphology. Based on this result, CaCl₂) was excluded from the formulation. Addition of glucose to the formulation as shown in FIG. 3C ((10:10:80) L-isoleucine/DPPC/Glucose at 70% (v/v) Ethanol:water) and FIG. 3D ((10:10:80) L-isoleucine/DPPC/Glucose at 80% (v/v) Ethanol:water) resulted in solid particles with low surface to volume ratio, which was not desired for pulmonary delivery, since these characteristics may inhibit dispersibility of the particles during inhalation.

### Example 4. In Vitro Release of Flavopiridol from L-Isoleucine/DPPC Microparticles

In vitro release experiments were performed to characterize release of flavopiridol from the ILDF particles by utilizing demonstrated release protocols. Briefly, 5 mg of ILDF particles were placed in 6×1.5 mL Eppendorf tubes containing 0.5 mL of 1×PBS/0.2% Tween-20 aqueous solution at 37° C. To establish sink conditions, tween-20 was added to ensure dissolution of flavopiridol in the release medium. At each selected time point, one vial was removed and centrifuged at 16,000 rpm for 10 min, and the supernatant was collected and filtered using a 0.2 μm PTFE syringe filter. The supernatant was analyzed at 358 nm using UV-Vis spectroscopy for determination of flavopiridol concentration. After all vials were analyzed for release of flavopiridol, the cumulative percentage of flavopiridol was calculated using eq. (4), by taking the summation of flavopiridol released up to a designated time, t, and then normalizing by the amount of flavopiridol loaded (m_{f}).

$$Cumulative Release ( \% ) = \frac{1}{{m}_{f}} {\sum }_{t = 0}^{t = {t}^{′}} {m}_{t} \qquad ( 4 )$$

The in vitro release profile described above was quantified using a 2-term model similarly to previously described methods that incorporates initial burst and Fickian diffusion release as shown in eq. (5).

$$\frac{{M}_{t}}{{M}_{∞}} = {θ}_{b} ( 1 - {e}^{- {k}_{b} t} ) + {θ}_{d} ( 1 - \frac{6}{{π}^{2}} {\sum }_{n = 1}^{∞} exp ( - \frac{{π}^{2} {n}^{2} {D}_{e} t}{{r}_{g}^{2}} ) ) \qquad ( 5 )$$

The first term represented the process of “burst release”, with k_{b} as the burst constant and θ_{b} as the contribution of burst release. This burst process was mostly described as interfacial diffusion, where drug that was located near or at the surface of the particle underwent rapid dissolution.¹⁹ The 2^{nd} term describes diffusion of drugs from spherical matrices under Fickian diffusion, where Dₑ was the effective diffusion coefficient, r_{g} was the geometrical mean particle radius, and θ_{d} was the contribution of diffusion release. The constraint, θ_{b}+θ_{d}=1, was used for mathematical completeness, representing individual contributions in the release mechanism. The in vitro release profile underwent regression analysis using nonlinear least squares algorithm in MATLAB (Math Works, USA).

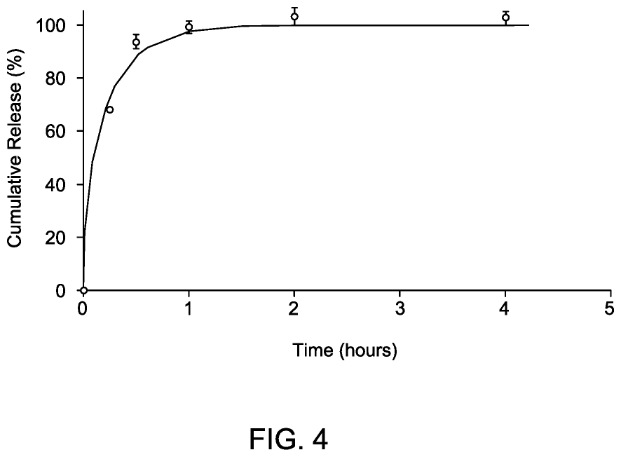

The overall release profile is shown in FIG. 4. By 3 hours, 99.4% of drug was released. In each time point of measurement, the result is shown as mean±SD from n=3 independent batches of ILDF microparticles. Nonlinear least-square fitting of the release profile using eq (5) is shown on FIG. 4 as the solid black line.

The quantitative kinetics was fitted using the kinetics model, described above and in eq. (5), and the extracted parameters are shown in Table 3. The in vitro release profile of flavopiridol matched desired release kinetics for pulmonary delivery.

TABLE 3
Kinetics parameters

| Parameter | Description | Result |
|---|---|---|
| K_{b} | Burst constant (hr−1) | 1.599 |
| 0b | Burst release fraction | 0.00186 |
| Dₑ | Diffusion coefficient (cm²/s) | 2.341E−11 |
| Rₚ | Radius of particle (cm) | 0.00025 |
| θd | Diffusion release fraction | 0.9981 |
| R² | Coefficient of determination | 0.9905 |

### Example 5. Stability Measurements of ILDF Particles

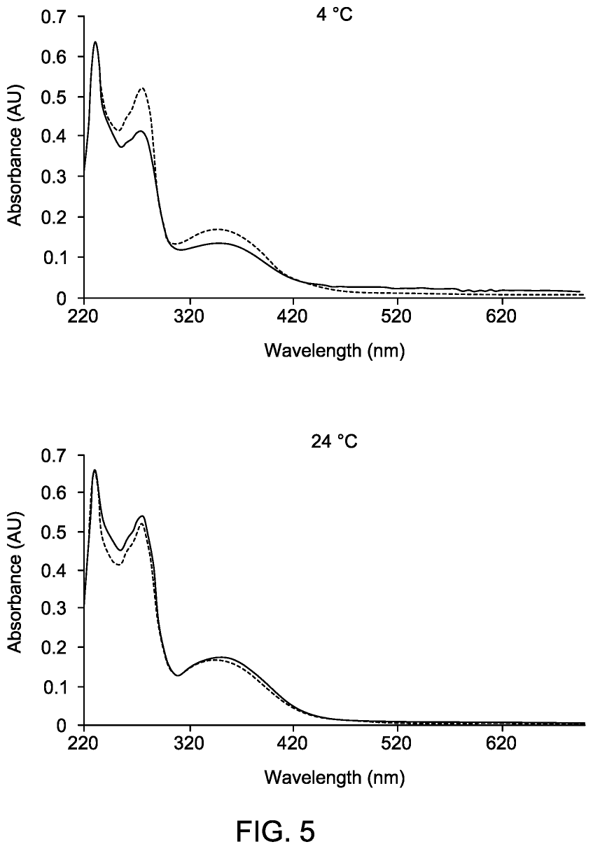

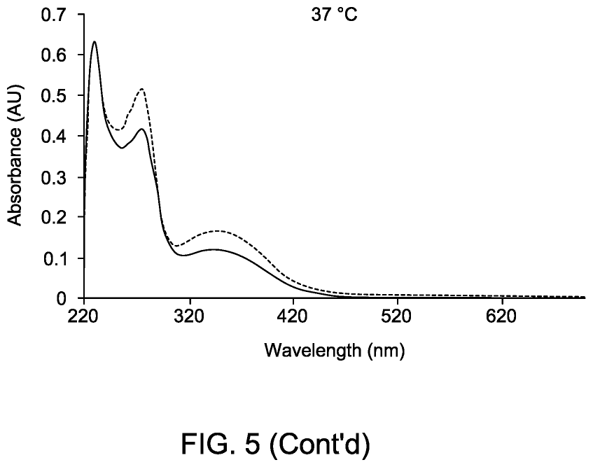

The stability of the ILDF particles (Table 2) was determined. 1-2 mg of freshly prepared particles described In Example 1 were placed in 24×1.5 mL Eppendorf tubes equally over four different environments. The four environments were 24° C. in the light and dark, 4° C. in the refrigerator, and 37° C. in an incubator. At time points, 0.5 mL of 70% ethanol was dispensed in the vial, and the contents were sonicated for 15 minutes until complete dissolution was reached. The solution was filtered into a quartz cuvette via 0.2 μm PTFE syringe filter and the UV-Vis spectrum was measured from 220-750 nm for each of the samples. The measured spectrums were compared qualitatively against freshly produced ILDF formulation to determine stability of the formulation. As shown in FIG. 5A, the stability under 4° C., mimicking the storage under house-hold refrigeration, the appearance of the spectrum of ILDF particles after 4 weeks closely resembled the spectrum of freshly produced particles. Under 24° C. and in the presence of room light, mimicking house-hold shelf storage, the stability remained throughout the 4 weeks of the test period as shown in FIG. 5B. Stability at 37° C. is also shown in FIG. 5C.

### Example 6. Attenuated Total Reflectance-Fourier Transform Infrared Spectroscopy

Attenuated Total Reflectance-Fourier Transform Infrared Spectroscopy (ATR-FTIR) was used to characterize the particle formulation for identification of components, including excipients and drugs. Briefly, <1 mg of freshly produced formulation particles were placed and compressed on the ATR diamond crystal. The FTIR spectrum was acquired using a Bruker Tensor 27 FTIR (Bruker Corporation, Billerica, MA). The spectrum was obtained over wavenumber range, 4000-400 cm⁻¹. The spectrum was averaged over 30 scans with a wavenumber resolution of 4 cm⁻¹ The individual components of the formulation particles such as flavopiridol, DPPC, and L-isoleucine were acquired to compare as a control.

### Example 7. In-Vitro Bioactivity Assay

The bioactivity of flavopiridol released from ILDF particles was determined by a luciferase reporter assay as described previously. ILDF particles (18.94 mg) containing 0.190% (w/w) flavopiridol was dissolved in DMEM with 10% FBS to a stock solution of 8.9×10⁴ nM flavopiridol. The bioactivity of flavopiridol was measured by its ability to suppress TNF-stimulated luciferase reporter expression, driven by a NF-kB responsive promoter. Human embryonic kidney 293 cells (HEK293) cells (ATCC) harboring a NF-kB-driven luciferase reporter were seeded in 96-well plates (10,000 cells/well in triplicates) 24 hours before the experiment. Cells were then treated with 100 μL of media containing 0.6 nM recombinant human TNF-α (Peprotech), in the presence or absence of various amount of dissolved flavopiridol formulation or blank formulation equivalent. After 24 h, 100 μL of 2× lysis buffer (Invitrogen) containing luciferase substrate was added directly to each well. Luminescence was measured in a plate reader (SpectraMax iD3, Molecular Devices). Experiments were performed in triplicate (N=3) from one batch of particles, and results were reported as mean±SD.

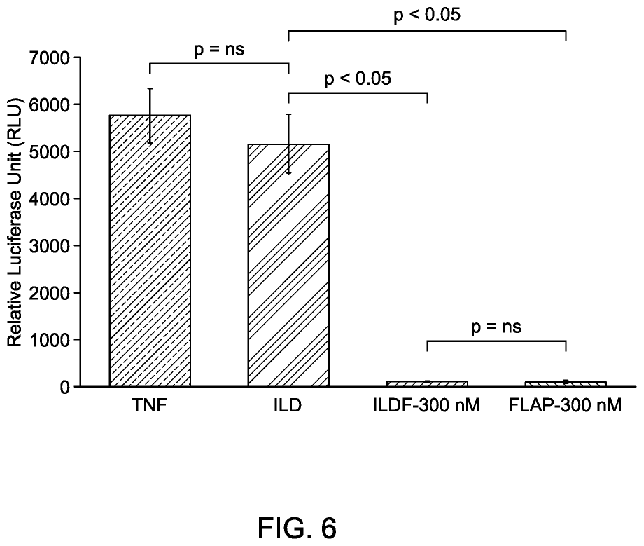

Results are shown in FIG. 6. The luminescence reading from samples treated with TNF-α only was arbitrarily set to 100% and then compared to flavopiridol-treated samples. The expected maximum luciferase activity was detected in cells treated with TNF-α only (black bar). The blank ILD particles (Grey bar) showed no statistical difference from the TNF-α only samples. In the presence of 300 nM of soluble unformulated flavopiridol, the luciferase activation was suppressed, Cells treated with 300 nM of flavopiridol in the ILDF particles (Red bar) also suppressed the luciferase activity and showed no statistical difference from the soluble flavopiridol. This result indicated that the biological activity of flavopiridol in the ILDF particles is retained.

### Example 8. The ILDF Particles Dispersity and Aerodynamic Properties

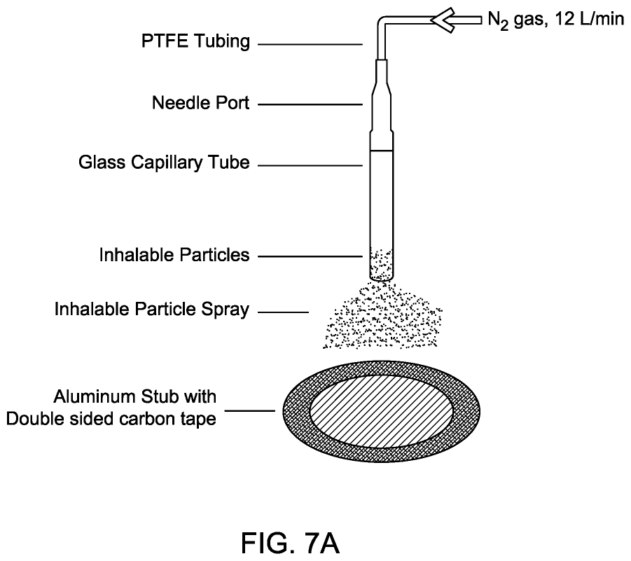

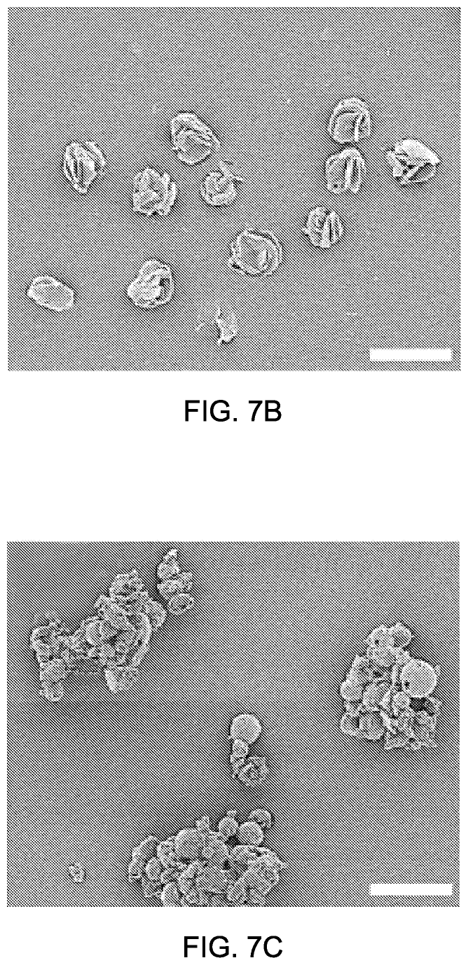

The dispersity of the ILDF formulation particles (Table 2) was assessed in a bench top assay mimicking the pulmonary inhalation process. Briefly, <1 mg of powder formulations were inserted into a 1.00 mm ID glass capillary tube and mounted vertically via 3-fingered clamps. The end of the capillary tube was custom fitted with a syringe-needle port, which is inserted into a PTFE tubing with the end of the PTFE tubing directly fitted to a variable area flowmeter attached to nitrogen gas tank (see schematic FIG. 7A). The flow rate of nitrogen was set at rates comparable to the human breath at sea level, at 12 L/min. Following the spray of the particles, the sample was prepped for SEM imaging as described in the experimental section. As shown in FIG. 7B, the ILDF particles exhibit high dispersity, with a mean distance between particles (x̅)=7.3±5.5 μm clearly resolved between particles. The physical diameter of the ILDF particles was measured from SEM images to be 5.5±1.3 μm. The mean aerodynamic diameter, measured according to the methods of Minne (Minne et al. (2008) European Journal of Pharmaceutics and Biopharmaceutics 70 (3), 839-844) was determined to be 2.5±0.6 μm, which falls into the desired aerodynamic range for pulmonary delivery of 1-5 μm. A summary of properties of is shown in Table 4.

TABLE 4
Properties of the ILD and flavopiridol loaded ILD particles for pulmonary delivery

| | Process | LC | EE | ρ_{tapped} | d_{g} | dₐ | |
|---|---|---|---|---|---|---|---|
| Formulation | yield | (%) | (%) | (mg/mL) | (μm) | (μm) | Dispersion |
| ILD-3 | 43 | 0 | 0 | 0.19 ± 0.01 | 5.4 ± 1.2 | 2.4 ± 0.5 | High |
| ILDF | 45 | 0.19 ± 0.01 | 99 ± 2 | 0.20 ± 0.01 | 5.5 ± 1.3 | 2.5 ± 0.6 | High |

To demonstrate the robustness of the bench top dispersity method, ILDG particles were synthesized using MPCA and parameter listed in Table 1, with D-α-glucose at 80% composition, and DPPC and L-isoleucine each at 10% to create sticky and cohesive particles. As shown in FIG. 7C, ILDG particles clumped together into aggregates of at least 20 particles, indicating little to no dispersibility.

### Example 9. Liquid Formulation for Inhalable Delivery

For a liquid formulation for inhalable delivery to the lung, flavopiridol was prepared. 15 mM Citric buffer was made by mixing 15 mM citric acid monohydrate (C₆H₈O₇·H₂O, formula weight (FW) 210.14) and 15 mM trisodium citrate dihydrate (C₆H—O₇Na₃·2H₂O, FW 294.12) to achieve a pH of 4-5. Flavopiridol free base (C₂₁H₂₀ClNO₅, FW 401.8) in powder form was weighed and dissolved in the buffer to reach 120 μM final concentration. The solution was measured by UV-Vis according to the methods of Example 2 over time points to assess stability. The formulated flavopiridol exhibited long-term (>3 months) stability.

For nebulization, a second formulation was prepared. 15 mM Citric buffer was made by mixing 15 mM citric acid monohydrate (C₆H₈O₇·H₂O. FW 210.14) and 15 mM trisodium citrate dihydrate (C₆H₅O₇Na₃·2H₂O, FW 294.12) solutions in a ratio to achieve pH between 4.0 to 5.0. Sodium chloride (NaCl, FW 58.5) was dissolved in the buffer to 70 mM NaCl concentration. Flavopiridol free base (C₂₁H₂₀ClNO₅, FW401.8) was weighed and dissolved in the buffer to reach 120 μM final concentration.

### Example 10. Administration of Flavopiridol in Lung Fibrosis Model

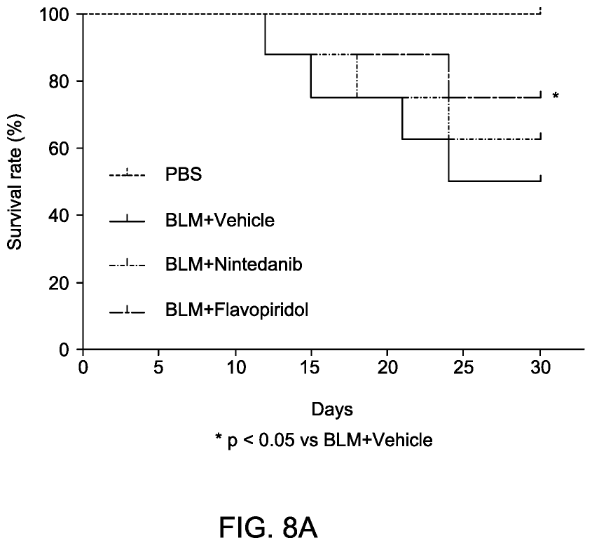

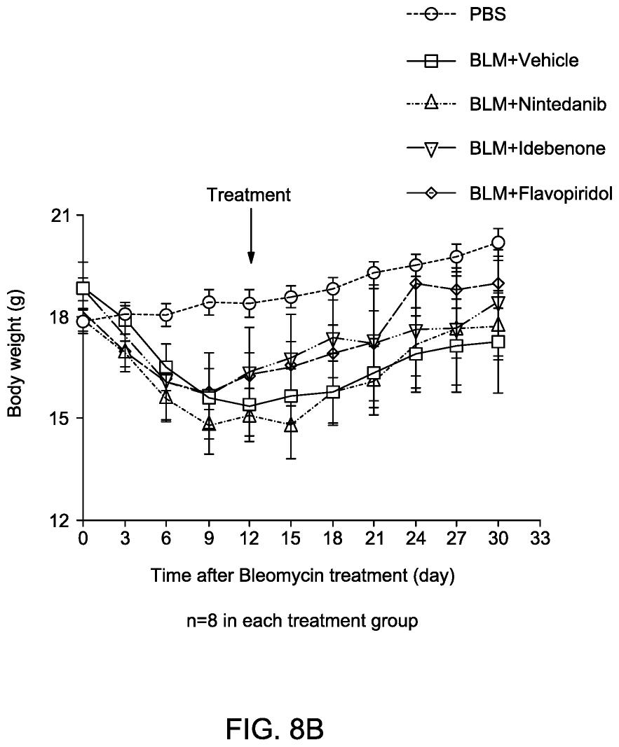

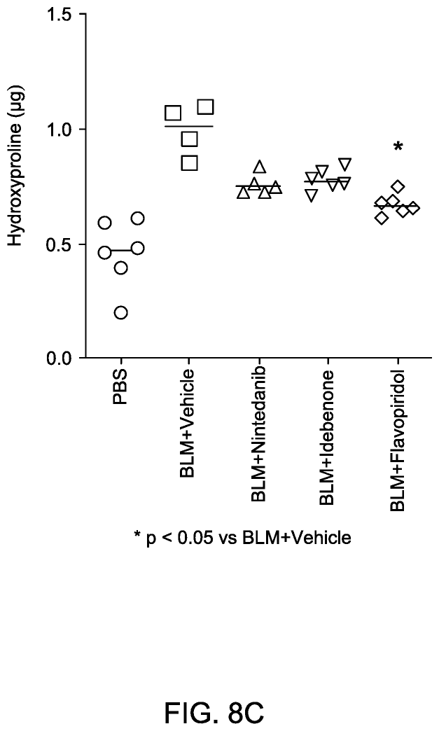

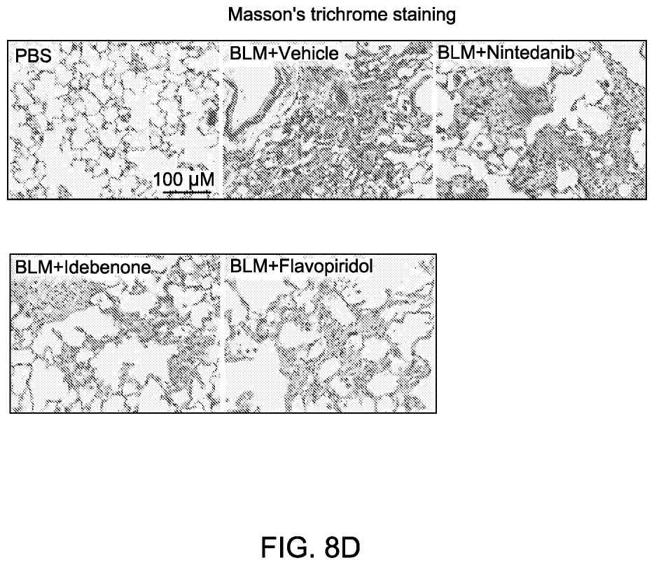

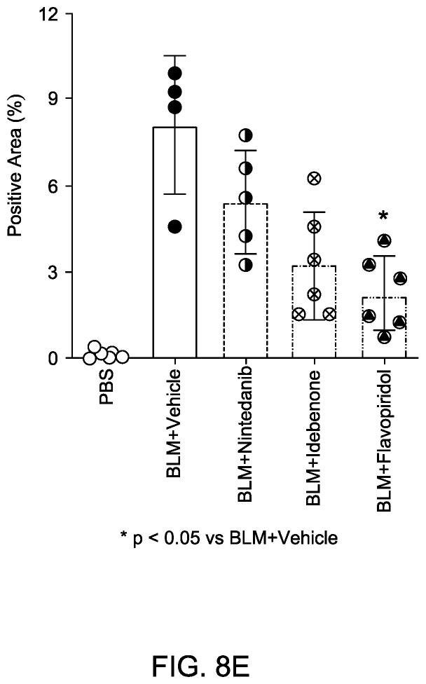

The effects of flavopiridol on lung inflammation and fibrosis were assessed in a lung fibrosis mouse model. C57B/L6 mice were treated with bleomycin (BLM) by microsprayer. At day 7 after BLM treatment, alveolar damage became apparent in the treated mice. At day 9, the mice were divided into 4 groups of 8 animals each. Each group received its corresponding treatment every other day starting at Day 9. At Day 30, the animals were sacrificed and assessed. One group received only phosphate buffer saline (PBS). One group received 28 mg/kg nitedanib by intraperitoneal injection (i.p.). One group received 2.5 mg/kg of flavopiridol i.p. and one group received 28 mg/kg idebenone i.p. Survival and lung morphology were assessed at day 30. Results are shown in FIGS. 8A-8E. The administration of flavopiridol showed a statistically significant difference in survival rate as compared to the BLM+ vehicle control (FIG. 8A). The flavopiridol treated mice generally maintained a higher body weight than the BLM+ vehicle control (FIG. 8B) and a statistically significant lower level of hydroxyproline (FIG. 8C). Lung tissue sections from the treated animals were stained with Masson's trichome staining protocol and exemplary images are shown in FIG. 8D. Quantitation of the staining is shown in FIG. 8E. The flavopiridol treated mice has a statistically significant decrease in percentage of stained area, indicating a reduction in the lung fibrosis in the flavopiridol treated mice as compared to the BLM+ vehicle control.

### Example 11. Formulation of Flavopiridol Hydrochloride Solution

Preparation of 15 mM Citrate Buffer. Weighed 176.46 mg Sodium Citrate Dihydrate and dissolved in 40 mL of Milli Q water to prepare 15 mM sodium citrate dihydrate solution. Weighed 115.272 mg citric acid and dissolved in 40 mL Milli Q water, to prepare 15 mM citric acid solution. Mixed 18 mL of 15 mM Sodium citrate Dihydrate solution and 28 mL of 15 mM citric acid solution to obtain 15 mM citrate buffer of pH 4.1

Preparation of 4.9 mM Flavopiridol hydrochloride Solution. 22.0 mg Flavopiridol hydrochloride was dispersed in 0.922 mL ethanol. To this dispersion transferred 9.322 mL 15 mM citrate buffer of pH 4.1. Dissolved the flavopiridol hydrochloride in above solvent by heating on water bath at 50° C. for 15 minutes. Flavopiridol solution was cooled down to room temperature and filtered through 0.22 μm membrane filter. Concentration of flavopiridol hydrochloride solution was measured using UV-Visible spectroscopy to confirm the 4.9 mM flavopiridol hydrochloride solution.

TABLE 5
Flavopiridol Hydrochloride solutions formulation

| 15 mM | | Flavopiridol | pH of the |
|---|---|---|---|
| Citrate buffer | Ethanol | HCL | solution |
| 9.322 mL (91%) | 0.922 mL (9%) | 22 mg (4.9 mM) | 4.1 |

### Example 12. Foaming Formulation of Flavopiridol Hydrochloride Solution

Preparation of 15 mM Citrate Buffer. Weighed 176.46 mg Sodium Citrate Dihydrate and dissolved in 40 mL of Milli Q water to prepare 15 mM Sodium Citrate Dihydrate solution. Weighed 115.272 mg citric acid and dissolved in 40 mL Milli Q water to prepare 15 mM citric acid solution. Mixed 18 mL of 15 mM Sodium citrate Dihydrate solution and 28 mL of 15 mM citric acid solution to obtain 15 mM citrate buffer of pH 4.1

Preparation of 4.9 mM Flavopiridol hydrochloride Solution. 22.0 mg Flavopiridol hydrochloride was dissolved in 10.243 mL 15 mM citrate buffer of pH 4.1 by heating on water bath at 50° C. for 15 minutes. Flavopiridol solution was cooled down to room temperature and filter through 0.22 μm membrane filter. Concentration of flavopiridol hydrochloride solution was measured using UV-Visible spectroscopy to confirm the 4.9 mM flavopiridol hydrochloride solution.

TABLE 6
Flavopiridol Hydrochloride solutions foaming formulation

| 15 mM Citrate buffer | Ethanol | Flavopiridol HCL | pH of the solution |
|---|---|---|---|
| 10.243 mL (100%) | 0 | 22 mg (4.9 mM) | 4.1 |

### Example 13. Local Delivery for Antifibrotic Therapy

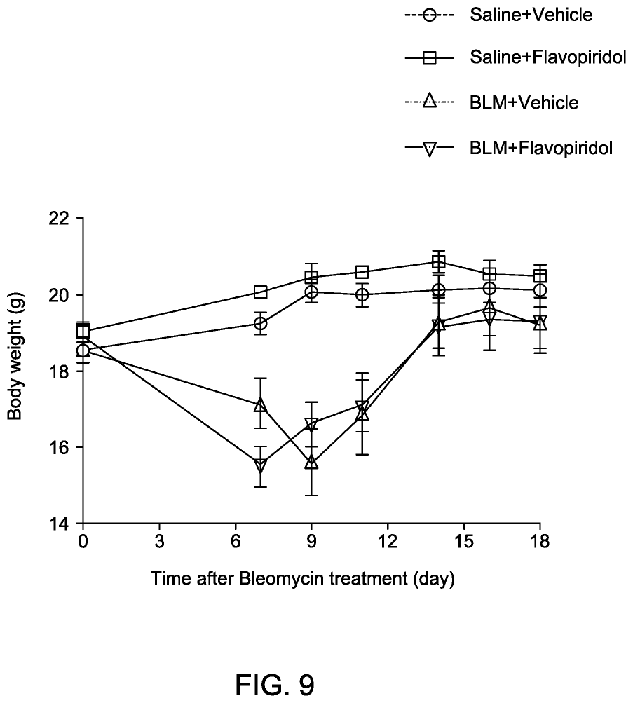

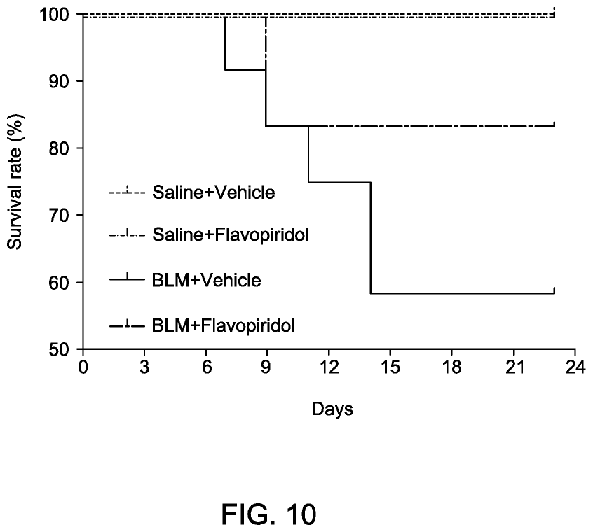

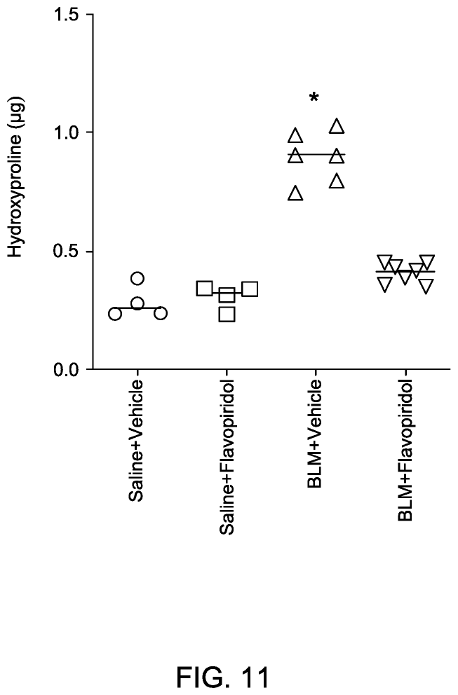

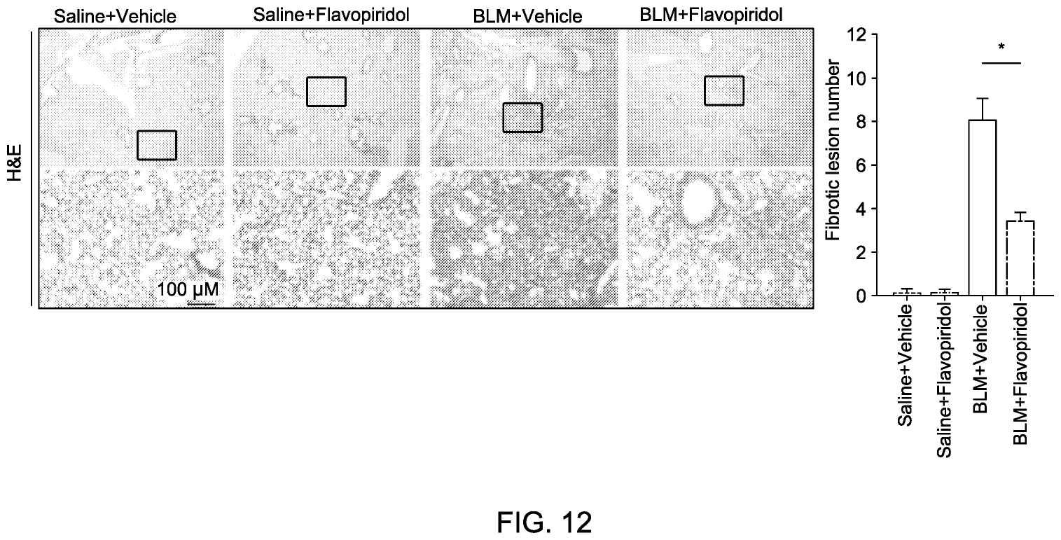

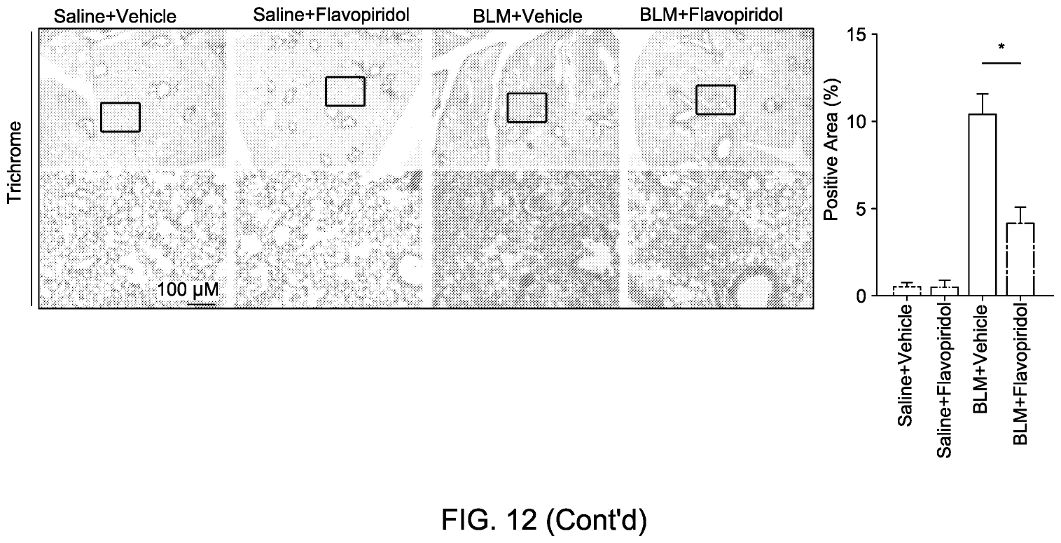

Our experimental model involved the induction of pulmonary fibrosis in mice using bleomycin, followed by the administration of inhaled flavopiridol during the early fibrotic phase of the model. Eight days post-intratracheal instillation of bleomycin, mice were treated with either a vehicle or inhaled flavopiridol every two days. After a 14-day treatment course, lung tissues were harvested and subjected to histological and biochemical analysis. A notable loss of body weight observed in bleomycin-exposed mice served as an indicator of successful fibrosis induction (FIG. 9). A higher survival rate was observed in the group of bleomycin-challenged mice treated with inhaled flavopiridol, compared to the vehicle group (FIG. 10). Furthermore, the treatment appeared to suppress the bleomycin-induced increase of hydroxyproline levels, a marker of fibrosis (FIG. 11).

Although the foregoing invention has been described in some detail by way of illustration and Example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A microparticle composition comprising a plurality of microparticles, wherein each microparticle comprises:
a hydrophobic amino acid or a hydrophobic peptide, or combinations thereof;
a lipid; and
a cyclin-dependent kinase 9 (CDK9) inhibitor.

2. The microparticle composition of claim 1, wherein the hydrophobic amino acid and/or hydrophobic peptide comprises alanine, valine, isoleucine, leucine, methionine, phenylalanine, tyrosine, tryptohan, L-isoleucine or combinations thereof.

3-6. (canceled)

7. The microparticle composition of claim 1, wherein the hydrophobic peptide comprises trileucine.

8. The microparticle composition of claim 1, wherein the lipid is a phosphocholine lipid.

9. The microparticle composition of claim 1, wherein the lipid is 1,2-diheptanoyl-sn-glycero-3-phosphocholine (DHPC), 1,2-dilauroyl-sn-glycero-3-phosphocholine (DLPC), 1,2-Dimyristoyl-sn-glycero-3-phosphocholine (DMPC), 1,2-dipalmitoyl-sn-glycero-3-phosphocholine (DPPC), or 1,2-distearoyl-sn-glycero-3-phosphocholine (DSPC), or combinations thereof.

10. (canceled)

11. The microparticle composition of claim 1, wherein the L-isoleucine is present in a ratio to the lipid of from 1000:1 to 1:10 (w/w), 100:1 to 1:1 (w/w), or about 9:1 (w/w).

12-13. (canceled)

14. The microparticle composition of claim 1, wherein the CDK9 inhibitor is flavopiridol, flavopiridol HCl, SNS-032, voruciclib, a pharmaceutically acceptable salt, or a derivative thereof, or a pharmaceutically acceptable salt thereof.

15-18. (canceled)

19. The microparticle composition of claim 1, wherein the lipid is present in a ratio to the CDK9 inhibitor of from 1000:1 to 1:1 (w/w), 100:1 to 10:1 (w/w), or 50:1 (w/w).

20-21. (canceled)

22. The microparticle composition of claim 9, wherein the L-isoleucine:DPPC:flavopiridol ratio is about 89.8:10.0:0.2 (w/w).

23. The microparticle composition of claim 1, wherein each microparticle has a mean geometric diameter of from 1 to 10 microns.

24. The microparticle composition of claim 1, comprising:
L-isoleucine;
DPPC (1,2-dipalmitoyl-sn-glycero-3-phosphocholine); and
flavopiridol, wherein the L-isoleucine:DPPC:flavopiridol ratio is about 89.8:10.0:0.2 (w/w).

25. The microparticle composition of claim 1, wherein the microparticle composition does not include CaCl₂) or glucose.

26. The microparticle composition of claim 1, consisting of:
L-isoleucine;
DPPC (1,2-dipalmitoyl-sn-glycero-3-phosphocholine); and
flavopiridol, wherein the L-isoleucine:DPPC:flavopiridol ratio is 89.8:10.0:0.19 (w/w).

27. The microparticle composition of claim 1, wherein the microparticle composition is a pharmaceutical composition used in treating inflammation or respiratory fibrosis in a subject in need thereof, wherein the microparticle composition comprises the cyclin-dependent kinase 9 (CDK9) inhibitor.

28. (canceled)

29. A method of preparing a plurality of particles for a microparticle composition, the method comprising:
sonicating a first reaction mixture comprising L-isoleucine and 1,2-dipalmitoyl-sn-glycero-3-phosphocholine (DPPC), water, and ethanol, wherein the L-isoleucine and DPPC are present in a ratio of about 9:1 (w/w), and wherein the ethanol:water ratio is about 70:30 (v/v), to prepare a 0.3% (w/v) feed mixture;
applying the feed mixture to a microfluidic piezo array to form a spray of micronized droplets; and
drying the droplets to afford the plurality of particles.

30. The method of claim 29, wherein the microfluidic piezo array is actuated at 113 kHz with 30V of power.

31. A liquid composition comprising:
a citric acid buffer having a pH of from 2 to 7; and
a cyclin-dependent kinase 9 (CDK9) inhibitor.

32. The liquid composition of claim 31, wherein the citric acid buffer comprises citric acid monohydrate and trisodium citrate dihydrate, having a pH of from 4 to 5, and wherein the CDK9 inhibitor is flavopiridol, SNS-032, voruciclib, or a derivative thereof, or pharmaceutically acceptable salt thereof.

33-35. (canceled)

36. The liquid composition of claim 31, wherein the cyclin-dependent kinase 9 (CDK9) inhibitor is at a concentration of about 120 μM.

37. The liquid composition of claim 36, further comprising sodium chloride.

38. The liquid composition of claim 37, comprising:
the citric acid buffer, having a pH of from 4 to 5;
flavopiridol at a concentration of about 120 μM; and
sodium chloride at a concentration of about 70 mM.

39. A method of administering a therapeutically effective amount of a composition comprising a CDK9 inhibitor or a CDKP inhibitor to a subject in need thereof, comprising administering to the subject a microparticle composition of claim 1, via respiratory administration.

40. A method of treating inflammation in a subject in need thereof, comprising administering a therapeutically effective amount of a liquid composition of claim 31, to the subject via respiratory administration, thereby treating the inflammation.

41. The method of claim 40, wherein the inflammation is respiratory inflammation, respiratory fibrosis, or lung fibrosis, and wherein the respiratory administration is inhalation administration or nasal administration.

42-48. (canceled)

49. The method of claim 41, further comprising administering an anti-fibrotic agent.

50. The method of claim 49, wherein the anti-fibrotic agent is pirfenidone, idebenone, nintedanib, Ifenprodil, n-acetyl cysteine, penetaxin, TD139, a corticosteroid, colchicine, D-penicillamine, pirfenidone (5-methyl-1-phenyl-2-[1H]-pyridone), interferon-β1a, relaxin, lovastatin, beractant, N-acetylcysteine, keratinocyte growth factor, captopril, hepatocyte growth factor, Rhokinase inhibitor, thrombomodulin-like protein, bilirubin, PPARγ (peroxisome proliferator-activated receptor gamma) activator, imatinib, or interferon-γ.

51. (canceled)
